(19) United States
(12) Patent Application Publication
(10) Pub. No.: US 2009/0029478 A1
(43) Pub. Date: Jan. 29, 2009

(54) DETECTION OF TARGET MOLECULES THROUGH INTERACTION WITH PROBES

(75) Inventor: Robert Steven Puskas, Manchester, MO (US)

Correspondence Address: WILSON SONSINI GOODRICH & ROSATI, 650 PAGE MILL ROAD, PALO ALTO, CA, 94304-1050, US

(73) Assignee: Singulex, Inc.

(21) Appl. No.: 11/621,928

(22) Filed: Jan. 10, 2007

Related U.S. Application Data
(63) Continuation of application No. 10/720,044, filed on Nov. 19, 2003, now abandoned.
(60) Provisional application No. 60/427,233, filed on Nov. 19, 2002.
(60) Provisional application No. 60/427,234, filed on Nov. 19, 2002.
(60) Provisional application No. 60/427,232, filed on Nov. 19, 2002.

(51) Int. Cl. G01N 33/00

(52) U.S. Cl. 436/94

(57) ABSTRACT

A method for detecting a target nucleic acid molecule or target nucleic acid molecular complex comprising: (a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and (b) detecting interaction of the probes with the molecule or molecular complex, said interaction being detected by an increase in fluorescence intensity during a detection interval having a fluorescence intensity above the fluorescence intensity of any individual free probe, wherein molecule or molecular complex is analyzed such that only individual molecules or molecular complexes in contact with a probe are within an interrogation volume and within a detection time interval.

### CROSS-REFERENCE

This application is a continuation of application Ser. No. 10/720,044, filed Nov. 19, 2003 which claims the benefit from Provisional Application Ser. Nos. 60/427,232, 60/427,233, and 60/427,234, each filed on Nov. 19, 2002, and each of which is incorporated herein by reference in its entirety.

### STATEMENT AS TO FEDERALLY SPONSORED RESEARCH OR DEVELOPMENT

Not Applicable.

### REFERENCE TO A SEQUENCE LISTING

The Sequence Listing, which is a part of the present disclosure, includes a text file comprising nucleotide and/or amino acid sequences of the present invention on a floppy disk. The subject matter of the Sequence Listing is incorporated herein by reference in its entirety.

### BACKGROUND OF THE INVENTION

I. Field of the Invention

The present invention relates to methods for identifying molecules, molecular interactions and molecular complexes by direct detection of interaction with one or more probes.

II. Description of the Related Art

1. Techniques for Detecting DNA Sequences

A variety of DNA hybridization techniques are available for detecting the presence of one or more selected polynucleotide sequences in a sample containing a large number of sequence regions. In a simple method, which relies on fragment capture and labeling, a fragment containing a selected sequence is captured by hybridization to an immobilized probe. The captured fragment can be labeled by hybridization to a second probe which contains a detectable reporter moiety.

Another widely used method is Southern blotting. In this method, a mixture of DNA fragments in a sample are fractionated by gel electrophoresis, then fixed on a nitrocellulose filter. By reacting the filter with one or more labeled probes under hybridization conditions, the presence of bands containing the probe sequence can be identified. The method is especially useful for identifying fragments in a restriction-enzyme DNA digest which contain a given probe sequence, and for analyzing restriction-fragment length polymorphisms (RFLPs).

Another approach to detecting the presence of a given sequence or sequences in a polynucleotide sample involves selective amplification of the sequence(s) by polymerase chain reaction. In this method, polymers complementary to opposite end portions of the selected sequence(s) are used to promote, in conjunction with thermal cycling, successive rounds of palmer-initiated replication. The amplified sequence may be readily identified by a variety of techniques. This approach is particularly useful for detecting the presence of low-copy sequences in a polynucleotide-containing sample, e.g., for detecting pathogen sequences in a body-fluid sample.

More recently, methods of identifying known target sequences by probe ligation methods have been reported. In one approach, known as oligonucleotide ligation assay (OLA), two probes or probe elements which span a target region of interest are hybridized with the target region. Where the probe elements match (basepair with) adjacent target bases at the confronting ends of the probe elements, the two elements can be joined by ligation, e.g., by treatment with ligase. The ligated probe element is then assayed, evidencing the presence of the target sequence.

In a modification of this approach, the ligated probe elements act as a template for a pair of complementary probe elements. With continued cycles of denaturation, reannealing and ligation in the presence of the two complementary pairs of probe elements, the target sequence is amplified geometrically, allowing very small amounts of target sequence to be detected and/or amplified. This approach is also referred to as Ligase Chain Reaction (LCR).

There is a growing need, e.g., in the field of genetic screening, for methods useful in detecting the presence or absence of each of a large number of sequences in a target polynucleotide. For example, as many as 150 different mutations have been associated with cystic fibrosis. In screening for genetic predisposition to this disease, it is optimal to test all of the possible different gene sequence mutations in the subjects genomic DNA, in order to make a positive identification of a “cystic fibrosis”. Ideally, one would like to test for the presence or absence of all of the possible mutation sites in a single assay.

These prior-art methods described above are not readily adaptable for use in detecting multiple selected sequences in a convenient, automated single-assay format. It is therefore desirable to provide a rapid, single-assay format for detecting the presence or absence of multiple selected sequences in a polynucleotide sample.

2. Primer Extension

One of the most powerful and versatile tools available to molecular biologists is the in vitro replication of nucleic acid sequences by primer extension, as exemplified by the ubiquitous techniques of polymerase chain reaction (PCR) and DNA sequencing. Both techniques include the steps of: 1) hybridizing a short, e.g. 15-30 nt, synthetic oligonucleotide primer to a single-stranded template nucleic acid; and 2) enzymatically extending from the 3, hydroxyl terminus of the primer in the presence of nucleotide 5′-triphosphates, complementary to the template strand, and a polymerizing enzyme. By this general primer extension method, sequencing information is generated, template nucleic acids are amplified or copied, and other genetic analysis tests are conducted. Results are optimized through the choice and concentrations of primers, multiple primers, enzymes, nucleotides, and other reagents, and the selection of temperature, temperature cycling conditions, and other experimental conditions. The choice of primers has been primarily limited to 2′-deoxyoligonucleotide primers made by the phosphoramidite chemistry method on automated synthesizers. Whereas nucleic acid analogs are known which efficiently hybridize to DNA or RNA, some with comparable or superior hybridization specificity and/or affinity, enzyme-mediated formation of a new phosphodiester bond only occurs between a primer having a 3′ terminal hydroxyl and a nucleotide having a 5′-triphosphate, or a closely related isostere, i.e., .alpha-thiotriphosphate, etc. Most structural permutations in either the primer or the nucleotide severely compromise the efficiency of primer extension, or negate it totally.

Nucleic acid analogs are structural analogs of DNA and RNA and which are designed to hybridize to complementary nucleic acid sequences. Through modification of the internucleotide linkage, the sugar, and/or the nucleobase, nucleic acid analogs may attain any or all of the following desired properties: 1) optimized hybridization specificity or affinity, 2) nuclease resistance, 3) chemical stability, 4) solubility, 5) membrane-permeability, and 6) ease or low costs of synthesis and purification.

3. Peptide Nucleic Acids (PNA)

A useful and accessible class of nucleic acid analogs is the family of peptide nucleic acids (PNA) in which the sugar/phosphate backbone of DNA or RNA has been replaced with acyclic, achiral, and neutral polyamide linkages. The 2-aminoethylglycine polyamide linkage in particular has been well-studied and shown to impart exceptional hybridization specificity and affinity when nucleobases are attached to the linkage through an amide bond.

2-Aminoethylglycine PNA oligomers typically have greater affinity, i.e. hybridization strength and duplex stability for their complementary PNA, DNA and RNA, as exemplified by higher thermal melting values (Tm), than the corresponding DNA sequences. The melting temperatures of PNA/DNA and PNA/RNA hybrids are much higher than corresponding DNA/DNA or DNA/RNA duplexes (generally 1.degree. C. per bp) due to a lack of electrostatic repulsion in the PNA-containing duplexes. Also, unlike DNA/DNA duplexes, the Tm of PNA/DNA duplexes are largely independent of salt concentration. The 2-aminoethylglycine PNA oligomers also demonstrate a high degree of base-discrimination (specificity) in pairing with their complementary strand. Specificity of hybridization can be measured by comparing Tm values of duplexes having perfect Watson/Crick complementarity and those with one or more mismatches. The degree of destabilization of mismatches, measured by the decrease in Tm (.DELTA.Tm), is a measure of specificity. In addition to mismatches, specificity and affinity are affected by structural modifications, hybridization conditions, and other experimental parameters. The neutral backbone of PNA also increases the rate of hybridization significantly in assays where either the target, template, or the PNA probe is immobilized on a solid substrate. Without any electrostatic repulsion, the rate of hybridization is often much higher for PNA probes than for DNA or RNA probes in applications such as Southern blotting, northern blots, or in situ hybridization experiments. Unlike DNA, PNA can displace one strand, “strand invasion”, of a DNA/DNA duplex. With certain DNA sequences, a second PNA can further bind to form an unusually stable triple helix structure (PNA).sub.2/DNA. PNA have been investigated as potential antisense agents, based on their sequence-specific inhibition of transcription and translation. PNA oligomers themselves are not substrates for polymerase as primers or templates, and do not conduct primer extension with nucleotides.

4. Linked Nucleic Acids (LNA)

Locked Nucleic Acids (LNA) monomers are bi-cyclic compounds structurally very similar to RNA-monomers, LNA share most of the chemical properties of DNA and RNA. However, introduction of LNA monomers into either DNA or RNA oligos results in unprecedented high thermal stability of duplexes with complementary DNA or RNA, while, at the same time obeying the Watson-Crick base-pairing rules.

5. Detection of DNA Mutations

Detection of specific genetic sequences is an area of active research and development. However, many problems still exist, such as low levels of signal, small sample size, high sample complexity, and the like. Improvements in the ability to provide a multiplicity of labels to a specific probe sequence are of interest, particularly using reagents that are compatible with standard phosphoramidite synthesis. The present invention addresses these issues.

The detection of mutations in sequences of DNA is becoming increasingly important in medical science. The detection of such a mutation in a DNA sequence typically involves the use of an oligodeoxyribonucleotide probe that is complementary to the target DNA sequence. The probe is designed to present some moiety, such as a radioactive element, that signals the occurrence of hybridization in a filter assay or an electrophoretic gel. The identification of hybridization has been used diagnostically for specific bacterial infections by detection of Mycobacterium tuberculosis genomic DNA, gonorrhea rRNA, Chiamydia genomic and plasmid DNA and Escherichia coli and Bacillus subtilis rRNA. Hybridization assays have also been developed for viral detection, including cytomegalovirus (CMV), human papilloma virus (HPV), and HIV-1.

By combining target amplification with allele specific oligonucleotides, small samples of human DNA can be analyzed for purposes of genetic screening, including the study of genetic changes associated with well-known inherited diseases. For instance, cancers typically display familial site-specific clustering. The identification of this kind of clustering can aid in the determination of enhanced risk for the development of the particular cancer. In addition, hereditary metabolic variations in DNA have been identified that affect the metabolism of known carcinogens. A variation that would increase the metabolism of a carcinogen may impact the likelihood of the development of cancer and, if developed, the speed of the cancer's growth.

6. Hybridization Probes

Traditional hybridization methods have been developed which employ radioactive probes with separation on filters. While radioactive probes have performed suitably well, growing concern over the use of radioactive materials has stimulated a search for alternative probes that achieve similar levels of sensitivity and performance without the risks and dangers associated with radioactive materials. For instance, biotin has been incorporated into an oligodeoxyribonucleotide for use in biotinavidin-linked analyses. In addition, numerous modifications of DNA have been used in the development of other alternative probes, including links to antibodies, gold-antibodies, mercury for double antibody reactions, eupsoralen, and fluorescent dye links for fluorescence detection of hybridization. These alternative methods typically allow approximately 10.sup.5 to 10.sup.6 copies of the DNA to be detected.

These and related advancements in the art have given rise to several methods of DNA mutation detection. These methods include denaturing gradient gel electrophoresis (DGGE), single-strand conformational polymorphisms (SSCP), temperature gradient gel electrophoresis (TGGE), the heteroduplex method (HET), ribonuclease cleavage, chemical cleavage of mismatch (CCU), ligase assay, allele-specific amplification (ASA) dideoxy fingerprinting (ddF), and allele-specific oligonucleotides (ASO). DGGE, SSCP, TGGE, HET, and ddF are frequently used to locate which exons of a gene contain mutations.

The currently available non-radioactive methods for detecting mutations in DNA have been somewhat problematic. For example, these methods have been generally unable to consistently provide accurate results in detecting point mutations in DNA. These detection methods have also proven to be time-consuming and quite costly to use. In addition, these non-radioactive mechanisms require a significant amount of DNA to perform their detecting function, though many times only a small quantity of DNA is available for analysis. Moreover, these methods are difficult to use, often requiring complex instruments and highly trained technicians not available in many laboratories. Finally, the materials utilized in these methods are generally either fragile or prone to degradation during the testing procedure.

Many detection techniques for nucleic acids utilize short, labeled oligonucleotide probes, containing a small number of labels. These have the advantage of being well defined, they have limited signal intensity due to the limited number of labels present in the probe. Several methods have been described to increase the length and number of labels within probes and correspondingly increase the probe intensity.

Belke et al. (WO 00/462342, PCT/US00/02897) describe an invention which provides novel nucleic acid labeling techniques that generate nucleic acid probes with specific activities at least ten fold higher than the levels obtained using standard labeling methods. Specifically, the methods of the invention provide methods of producing nucleic probes that each comprises multiple labeled nucleotides. The methods can be used to generate RNA, DNA or hybrid probes. The invention also provides reaction mixtures and kits for the practice of the methods of the invention and compositions comprising the probes generated according to the methods of the invention. The probes described in this method can be either double stranded or single stranded and contain a signal domain ranging from 5 to 100 nucleotides in which the label is incorporated.

In another example the probes are primarily double stranded. The application of Shafer (WO 00/04192, PCT/US99/16242) describe gene probes including a number of related designs for gene probe components, multilinking components and signaling components, all of which are modular in nature and can be used together or in part. These components are generally joined together in composite structures by hybridization of complementary sub-segments, called linkers. The reporters of the present invention are also designed to be conjoined into arrays that can provide amplified signaling. The multilinking components of the present invention may be interposed between the probe and the reporter units and provide for the binding of multiple reporters. These probe and signaling methods also include means to achieve mixed-color labeling that is specific to each target. The probes are useful for detecting target sequences in a wide variety of formats including, but not limited to, membrane formats, in situ formats, and on various solid substrate chip formats. However, these methods generally result in either relatively short labeled probes or multimeric probes of varied size and intensity.

Several methods have been described for generating highly labeled primers using synthetic branched structures including Urdea et al. (U.S. Pat. No. 5,681,697), Mandrand et al. (U.S. Pat. No. 5,695,936) and Gryaznov (U.S. Pat. No. 5,571,677) which require complex synthesis methods. In addition the resulting probes are likely to have varied size and intensity.

Using the techniques illustrated above and other described methods, probes have been constructed for the detection of nucleic acids, and applied in a variety of different and increasingly more sensitive detection methods for individual or multiple nucleic acid targets. However, a need exists for materials and methods which will enable analysis of nucleic acids (and especially multiple nucleic acid targets) at extremely low levels. Two means that can help accomplish this are solution hybridization, which maximizes hybridization effectiveness and single molecule detection for maximal sensitivity. This invention describes the production and use of highly labeled probes of unitized size, high intensity, charge, and mass for use in hybridization, but especially for use in single molecule detection and molecular electrophoresis.

### BRIEF SUMMARY OF THE INVENTION

Accordingly, it is an object of the invention to overcome these and other problems associated with the related art. These and other objects, features and technical advantages are achieved by providing methods of detecting single molecules by interaction with probes.

Accordingly one aspect of the invention provides a method for detecting a target nucleic acid molecule or target nucleic acid molecular complex comprising: (a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and (b) detecting interaction of the probes with the molecule or molecular complex, said interaction being detected by an increase in fluorescence intensity during a detection interval having a fluorescence intensity above the fluorescence intensity of any individual free probe, wherein molecule or molecular complex is analyzed such that only individual molecules or molecular complexes in contact with a probe are within an interrogation volume and within a detection time interval.

Another aspect provides a method for detecting a target nucleic acid molecule or target nucleic acid molecular complex comprising: (a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and (b) detecting interaction of the probes in contact with the molecule or molecular complex, said interaction being detected by an increase in fluorescence intensity during a detection interval having a fluorescence intensity above the fluorescence intensity of any individual free probe; and (c) detecting the velocity of the probes in contact with molecule or molecular complex, in relation to an expected velocity for such a complex in a transport tube, wherein the velocity is imparted on the probes in contact with the molecule or molecular complex by pumping the probes in contact with the molecule or molecular complex through the tube or by applying an electric field to the probes in contact with the molecule or molecular complex.

Another aspect provides a method for detecting a target nucleic acid molecule or target nucleic acid molecular complex comprising: (a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and (b) detecting interaction of the probes with the molecule or molecular complex, said interaction being detected by a change during a detection interval in a fluorescence parameter selected from the group consisting of fluorescence lifetime, fluorescence polarization or FRET, wherein molecule or molecular complex is analyzed such that only individual molecules or molecular complexes in contact with a probe are within an interrogation volume and within a detection time interval.

Another aspect provides a method for detecting a target nucleic acid molecule or target nucleic acid molecular complex comprising: (a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and (b) detecting interaction of the probes in contact with the molecule or molecular complex, said interaction being detected by a change during a detection interval in a fluorescence parameter selected from the group consisting of fluorescence lifetime, fluorescence polarization or FRET, during a detection interval having a fluorescence intensity above the fluorescence intensity of any individual free probe; and (c) detecting the velocity of the probes in contact with molecule or molecular complex, in relation to an expected velocity for such a complex in a transport tube, wherein the velocity is imparted on the probes in contact with the molecule or molecular complex by pumping the probes in contact with the molecule or molecular complex through the tube or by applying an electric field to the probes in contact with the molecule or molecular complex.

Another aspect provides a method for determining the number of probes interacting with a target comprising: (a) contacting two or more probes that interact specifically with the target, said target being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex, and wherein fluorescent intensity of each probe is equal to that of other probes within detection capabilities of an instrument used for the detection; and (b) detecting interaction of the probes in contact with the target, said interaction being during a detection interval that is above the fluorescence intensity for any individual free probe molecule; (c) detecting the velocity of the probes in contact with the target, in relation to an expected velocity for such a complex in a transport tube, wherein the velocity of the molecular probes-target hybrids matches the expected velocity for such a complex in a transport tube, and wherein the velocity is imparted on the probes in contact with the molecule or molecular complex by pumping the probes in contact with the molecule or molecular complex through the tube or by applying an electric field to the probes in contact with the molecule or molecular complex; and (d) determining the number of probes interacting with each target molecule by dividing the fluorescent intensity detected per detection time interval by the unit intensity per detection time interval.

Another aspect provides a method for detecting a target nucleic acid molecule or target nucleic acid molecular complex comprising: (a) contacting two or more probes that interact specifically with the target, said probe species being labeled with one or more fluorescent dye molecules of the same dye or labeled with one or more dye molecules of at least one other dye that is indistinguishable from the first dye by their emission characteristics in the assay instrument, wherein probe labeling takes place to the extent that luminescent or fluorescent intensity per detection time unit of each probe species is unique, and wherein each probe species is differentiable from the other probe species in the assay based on luminescent or fluorescent intensity per detection time unit; and (b) detecting interaction of the probes in contact with the target, such that only individual target or probe molecules are within an interrogation volume and within a detection time interval and no more then one probe molecule is in the interrogation volume within a detection time interval, wherein specific probe species with target is detected by an increase in fluorescence intensity during a detection interval that is equal to the additive fluorescence intensities of more than one probe species; or (c) detecting interaction of the probes in contact with the target such that only individual target or probe molecules are within an interrogation volume and within a detection time interval and no more than one probe molecule is in the interrogation volume within a detection time interval, wherein specific multiple probe species with target is detected by an increase in fluorescence intensity during a detection interval that is equal to the additive fluorescence intensities of more than one probe species and the velocity of the molecular probes-target hybrids matches the expected velocity for such a complex in a transport tube, such molecular velocity imparted by pumping of sample through the tube or by application of an electric field to the sample.

These and other features, aspects and advantages of the present invention will become better understood with reference to the following description, examples and appended claims.

### BRIEF DESCRIPTION OF THE SEVERAL VIEWS OF THE DRAWINGS

FIG. 1. Intensity differentials using multiple different probes.

FIG. 2. Schematic diagram of the basic apparatus for single molecule detection using laser induced fluorescence.

FIG. 3. Heart of the SMD Instrument showing glass capillary tube.

FIG. 4. PCR products (approximately 0.1 ng/μl dye-labeled PCR product with and without 10 ng/μl unlabeled PCR product) were denatured at elevated pH for five minutes at room temperature, chilled on ice for five minutes, and adjusted to neutral pH to enable hybridization. Hybridized and re-annealed samples were diluted 1/1000 in 100 mM glycylglycine, 0.1% hydroxymethylpropyl cellulose, pH 8.2, and 50 ul of sample was loaded into a coated 200 um glass capillary. Electrophoresis was for five minutes at 2000 volts (constant). Photon cross correlation data were obtained by excitation of the sample with 635 nm lasers at 1 mW power, and detection at appropriate wavelength to detect fluorescence from the dye. The current was approximately 80 amperes. Approximately 1,000 cross correlation events were detected in each experiment. Cross correlation data represents cross correlation of photon bursts weighted by an intensity factor. Consequently brighter cross correlation for an equal number of cross correlation events will produce a peak with increased amplitude. The labeled PCR product alone was detected at approximately 650 msec (elapsed time between detection at the two detector positions). In the presence of excess unlabeled PCR product the peak at 650 msec was present, but it was reduced in intensity (decreased peak amplitude) due to hybridization with its unlabeled complement (FIGS. 4a and 4b).

FIG. 5. Photon bursts were analyzed using the instrument software to group those bursts generated by molecules. FIGS. 5a and 5b show a time course segment of the data for molecule photon bursts. Labeled-unlabeled hybrid molecules have photon bursts of 50 photons—photon bursts of 100 are seen with labeled-only molecules.

FIG. 6. Histogram of photon intensities of hybridized unitized probe. Annealed samples (see FIGS. 4a and 4b) were diluted 1/10,000 in 100 mM glycylglycine, 0.1% hydroxymethylpropyl cellulose, pH 8.2 and pumped into a coated capillary at 1 μl/minute for 3-5 minutes. Excitation was with 635 nm lasers at 1 mW, and detection was at the appropriate wavelength to detect fluorescence. Photon bursts were analyzed using the instrument software to group photon bursts into those groups of photons originating from individual molecules. Looking at time plots of the molecule photon burst intensities (FIG. 5) we see that labeled-unlabeled hybrid molecules typically have burst intensities of about 50 photons, while labeled-only molecules have photon bursts of about 100. Histograms of number of molecules vs. photon intensity were plotted (FIGS. 6a and 6b). With uniform illumination, identically-labeled individual molecules will produce equivalent photon bursts. The label-only molecules will be twice as bright (produce larger photon bursts) compared to the labeled-unlabeled hybrids. However, because the molecules are illuminated here by a non-uniform beam, the detectable photon bursts, when looked at in composite, have a distribution of intensities. Nonetheless, we can see from the molecule photon bursts (FIG. 6) that labeled-unlabeled hybrid molecules (1×Intensity) have photon bursts of about 50 photons, while the labeled-only molecules (2×Intensity) have photon burst sizes of about 100 photons. Using a cut-off point of 50 photons we also can see the overall distribution of 2×I molecules in the histogram of molecule intensities and can determine the number of labeled-unlabeled hybrid molecules detected in the experiment.

### DETAILED DESCRIPTION OF THE INVENTION

### I. Abbreviations and Definitions

To facilitate understanding of the invention, a number of terms and abbreviations as used herein are defined below as follows:

The term “label” refers to a moiety that, when attached to the compositions of the invention, render such compositions detectable using known detection means, e.g., spectroscopic, photochemical, radioactive, biochemical, immunochemical, enzymatic or chemical means. Exemplary labels include but are not limited to fluorophores, chromophores, radioisotopes, spin labels, enzyme labels and chemiluminescent labels. Such labels allow direct detection of labeled compounds by a suitable detector, e.g., a fluorescence detector. In addition, such labels include components of multi-component labeling schemes, e.g., a system in which a ligand binds specifically and with high affinity to a detectable anti-ligand, e.g., a labeled antibody binds to its corresponding antigen.

“Linking group” means a moiety capable of reacting with a “complementary functionality” to form a “linkage.” A linking group and its associated complementary functionality is referred to herein as a “linkage pair.” Preferred linkage pairs include a first member selected from the group isothiocyanate, sulfonyl chloride, 4,6-dichlorotriazinyl, succinimidyl ester, or other active carboxylate, and a second member that is amine. Preferably a first member of a linkage pair is maleimide, halo acetyl, or iodoacetamide whenever the second member of the linkage pair is sulfhydryl. (e.g., R. Haugland, Molecular Probes Handbook of Fluorescent Probes and Research Chemicals, Molecular probes, Inc. (1992)). In a particularly preferred embodiment, the first member of a linkage pair is N-hydroxysuccinimidyl (NHS) ester and the second member of the linkage pair is amine, where, to form an NHS ester, a carboxylate moiety is reacted with dicyclohexylcarbodiimide and N-hydroxysuccinimide.

The term “Watson/Crick base-pairing” refers to a pattern of specific pairs of nucleotides, and analogs thereof, that bind together through sequence-specific hydrogen-bonds, e.g. A pairs with T and U, and G pairs with C.

The term “nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine, (e.g., Komberg and Baker, DNA Replication, 2nd Ed. (Freeman, San Francisco, 1992)). The term “nucleotide” as used herein refers to a phosphate ester of a nucleoside, e.g., a triphosphate ester, wherein the most common site of esterification is the hydroxyl group attached to the C-5 position of the pentose. The term “nucleoside/tide” as used herein refers to a set of compounds including both nucleosides and nucleotides.

The term “polynucleotide” means polymers of nucleotide monomers, including analogs of such polymers, including double and single stranded deoxyribonucleotides, ribonucleotides, .alpha.-anomeric forms thereof, and the like. Monomers are linked by “internucleotide linkages,” e.g., phosphodiester linkages, where as used herein, the term “phosphodiester linkage” refers to phosphodiester bonds or bonds including phosphate analogs thereof, including associated counterions, e.g., H.sup.+, NH.sub.4.sup.+, Na.sup.+, if such counterions are present, Whenever a polynucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, and “T” denotes deoxythymidine, unless otherwise noted.

“Analogs” in reference to nucleosides/tides and/or polynucleotides comprise synthetic analogs having modified nucleobase portions, modified pentose portions and/or modified phosphate portions, and, in the case of polynucleotides, modified internucleotide linkages, as described generally elsewhere (e.g., Scheit, Nucleotide Analogs (John Wiley, New York, (1980); Englisch, Angew. Chem. Int. Ed. Engl. 30:613-29 (1991); Agrawal, Protocols for Polynucleotides and Analogs, Humana Press (1994)). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H.sup.+, NH.sub.4.sup.+, Na.sup.+, if such counterions are present. Exemplary modified nucleobase portions include but are not limited to 2,6-diaminopurine, hypoxanthine, pseudouridine, C-5-propyne, isocytosine, isoguanine, 2-thiopyrimidine, and other like analogs. Particularly preferred nucleobase analogs are iso-C and iso-G nucleobase analogs available from Sulfonics, Inc., Alachua, Fla. (e.g., Benner, et al. U.S. Pat. No. 5,432,272). Exemplary modified pentose portions include but are not limited to 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, bromo and the like. Modified internucleotide linkages include phosphate analogs, analogs having achiral and uncharged intersubunit linkages (e.g., Sterchak, E. P., et al., Organic Chem, 52:4202 (1987)), and uncharged morpholino-based polymers having achiral intersubunit linkages (e.g., U.S. Pat. No. 5,034,506). A particularly preferred class of polynucleotide analogs where a conventional sugar and internucleotide linkage has been replaced with a 2-aminoethylglycine amide backbone polymer is peptide nucleic acid (PNA) (e.g., Nielsen et al., Science, 254:1497-1500 (1991); Egholm et al., J. Am. Chem. Soc., 114: 1895-1897 (1992)).

As used herein the term “primer-extension reagent” means a reagent comprising components necessary to effect an enzymatic template-mediated extension of a polynucleotide primer. Primer extension reagents include (1) a polymerase enzyme, e.g., a thermostable DNA polymerase enzyme such as Taq polymerase; (2) a buffer; (3) one or more chain-extension nucleotides, e.g., deoxynucleotide triphosphates, e.g., deoxyguanosine 5′-triphosphate, 7-deazadeoxyguanosine 5′-triphosphate, deoxyadenosine 5′-triphosphate, deoxythymidine 5′-triphosphate, deoxycytidine 5′-triphosphate; and, optionally in the case of Sanger-type DNA sequencing reactions, (4) one or more chain-terminating nucleotides, e.g., dideoxynucleotide triphosphates, e.g., dideoxyguanosine 5′-triphosphate, 7-deazadideoxyguanosine 5′-triphosphate, dideoxyadenosine 5′-triphosphate, dideoxythymidine 5′-triphosphate, and dideoxycytidine 5′-triphosphate.

“Mobility-dependent analysis technique” means an analysis technique based on differential rates of migration between different analyte species. Exemplary mobility-dependent analysis techniques include electrophoresis, chromatography, sedimentation, e.g., gradient centrifugation, field-flow fractionation, multi-stage extraction techniques and the like.

A “target nucleic acid” sequence for use with the present invention may be derived from any living, or once living, organism, including but not limited to prokaryote, eukaryote, plant, animal, and virus. The target nucleic acid sequence may originate from a nucleus of a cell, e.g., genomic DNA, or may be extranuclear nucleic acid, e.g., plasmid, mitrochondrial nucleic acid, various RNAs, and the like. The target nucleic acid sequence may be first reverse-transcribed into cDNA if the target nucleic acid is RNA. Furthermore, the target nucleic acid sequence may be present in a double stranded or single stranded form.

A “probe” refers to a biopolymer comprising a recognition (R) moiety and tail (T) moiety of the general form R-B-T where B is a bridge connecting the recognition and tail moieties. A labeled probe further comprises one or more detectable label moieties covalently or non-covalently attached to or incorporated in the tail. The recognition moiety is a nucleic acid sequence which complementary to a nucleic acid sequence in the target molecule. The tail moiety is a defined length polynucleotide which may or may not include labels and/or mass tags.

“Detected unit”—Any detectable signal for a parameter. Detected units may be fluorescence, etc. . . . temporally or spatially determined. This is a dually digital system. Individual probes and probe-target complexes are detected (digital) and probes have a digital parameter unit (or subunit).

Parameter unit. A measure of a parameter which is discernable above the background measurement level of the analytical system, and which is associated with a specific, unitized probe (molecule), and which is designated as the base unit of measurement for the specific, unitized probe for that parameter. A probe may possess more than one type of parameter unit. A probe may have parameter units or subunits for more than one parameter. Measurement of more than one parameter may be used to discern (identify) a specific unitized probe or probe target. Probes detected simultaneously (by measurement of a parameter) can be discriminated from one another (or by inference associated with the target molecule) by measuring one or more other parameters (parameter units) possessed by one or more of the detected probes. A parameter of the target also may be used to distinguish between probe-targets and probes. (The parameter unit for a probe also may be tied to a given set of conditions, e.g. velocity in our instrument system).

It is of interest to maximize the detection of analytes for increased sensitivity. The ultimate detection level is that of detecting individual molecules, interactions between molecules and molecular complexes. The following invention details methods for detecting such molecules, molecular interactions or molecular complexes particularly by means of flow cytometry, single molecule electrophoresis or other single-molecule analytical instrumentation.

Single molecule detection is the ultimate limit for detection. Within the field of single molecule detection, optical-based detection systems (i.e. laser-induced fluorescence) are generally considered the most desirable option currently available for detection of biochemicals. Optical microscopy was used in the first published documentation of single molecule detection (Hirschfeld 1976). Since then, the field of optical detection of single molecules has been rapidly advanced by a variety of improvements in methodology.

A typical system used for detection in fluids involves a fluid flow system similar to flow cytometry systems in which fluorescence from a sample stream is measured as the stream passes a detector. These methods have been applied for sizing of DNA fragments (Ambrose et al. 1993; Castro et al. 1993; Goodwin et al. 1993a; Huang et al. 1996; Johnson et al. 1993; Larsen et al. 2000; Petty et al. 1995), for progress toward single molecule DNA sequencing (Ambrose et al. 1993; Davis et al. 1991; Goodwin et al. 1995; Goodwin et al. 1996; Goodwin et al. 1993b; Harding and Keller 1992; Jett et al. 1989; Soper et al. 1991a; Van Orden et al. 2000; Werner et al. 2003), and to detect DNA hybridization via two-color fluorescence (Castro and Williams 1997). A variation of the fluid flow technology involves application of an electric field in which molecules move electrophoretically (Castro and Al. 1995; Castro and Shera 1995; Chen and Dovichi 1996; Haab and Mathies 1995; Ma et al. 2001; Shortreed et al. 2000; Soper et al. 1995b; Van Orden and Keller 1998) and which has been utilized for sizing of DNA. Electrophoretic single molecule analysis (molecular electrophoresis) also has been used to detect protein-DNA interactions via two-color fluorescence (LeCaptain et al. 2001). To date no system has combined the differentiating capabilities and single molecule sensitivity of molecular electrophoresis for multiplexed hybridization detection.

Systems capable of single-molecule detection use a single laser or laser beam to detect down to single molecules on surfaces or in solution. Fluorescence correlation spectroscopy and other confocal instrumentation interrogate minute volumes, consequently have limited molecular throughput, and are not suitable for detecting very low levels of analytes (Nie and Zare 1997). Atomic Force Microscopy can detect single molecules, but it also suffers from limited molecular throughput (Wrotnowski 2002). These systems also have limited potential for multiplexing. Our single molecule electrophoresis instrument offers single molecule detection, high specificity, high molecular throughput and extensive multiplexing capabilities.

### II. Single Molecule Detection System

Our basic method for ultrasensitive detection relies on single-molecule fluorescence analysis. A sample to be analyzed is first obtained from air, water, surfaces or tissue. No polymerases, enzymes or proteins, or any amplification processes are necessary so sample preparation times and complexity are minimal. This single molecule detection instrument provides an ultrasensitive means to reliably detect individual molecules.

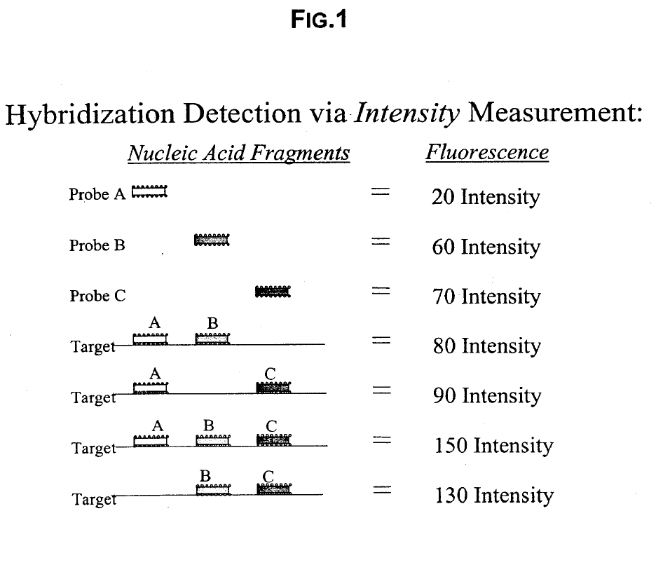

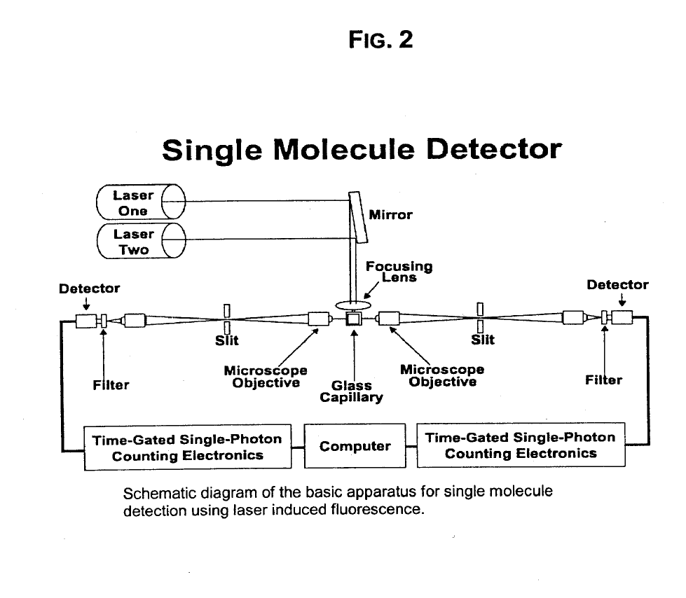

The basic detection scheme is shown in FIG. 1. The apparatus consists of two lasers (or a single laser source split into two beams), focusing light-collection optics, two single photon detectors, and detection electronics under computer control. A sample compartment (not shown) contains two reservoirs that hold the solution being analyzed. The reservoirs are connected by tubing to a glass capillary cell that is square in cross section.

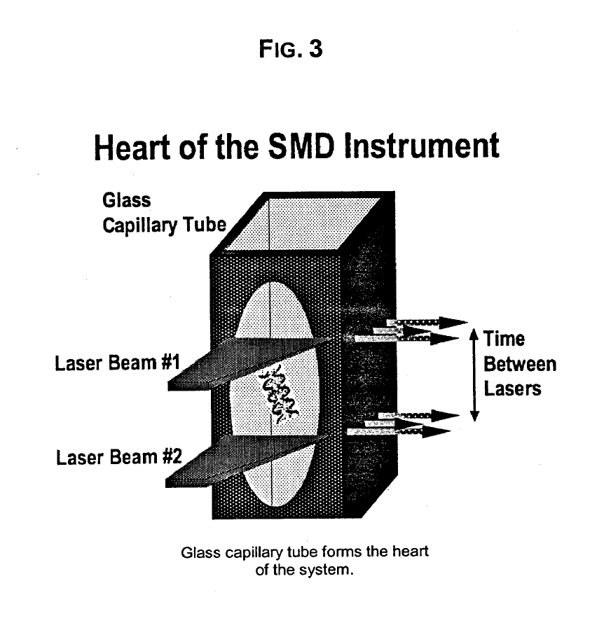

The heart of the system is the glass capillary flow cell of the apparatus shown in FIG. 2. Two laser beams (5 μm in diameter) are optically focused about 100 μm apart perpendicular to the length of the sample-filled capillary tube. The lasers are operated at particular wavelengths depending upon the nature of the detection probe to be excited. The interrogation volume of the detection system is determined by the diameter of the laser beam and by the segment of the laser beam selected by the optics that direct light to the detectors. The interrogation volume is set such that, with an appropriate sample concentration, single molecules (single nucleic acid probes or single probe-target hybrids) are present in the interrogation volume during each time interval over which observations are made.

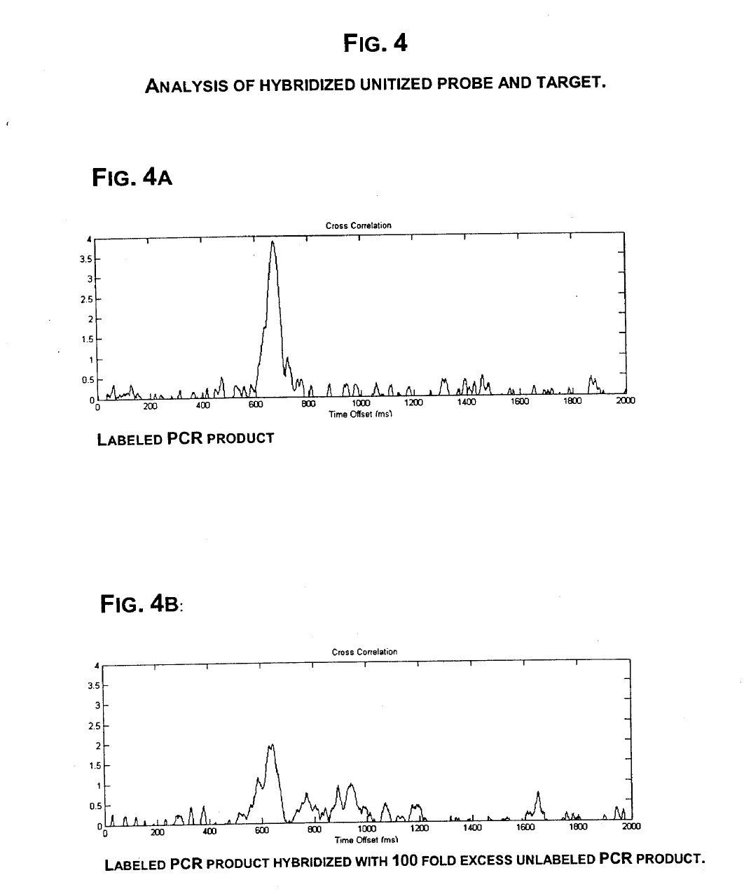

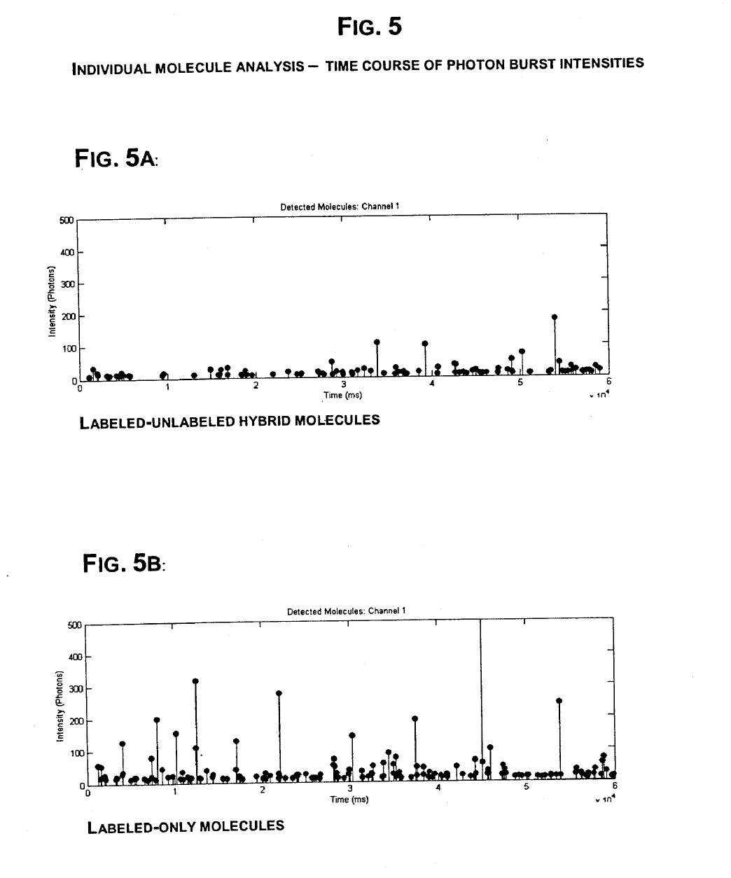

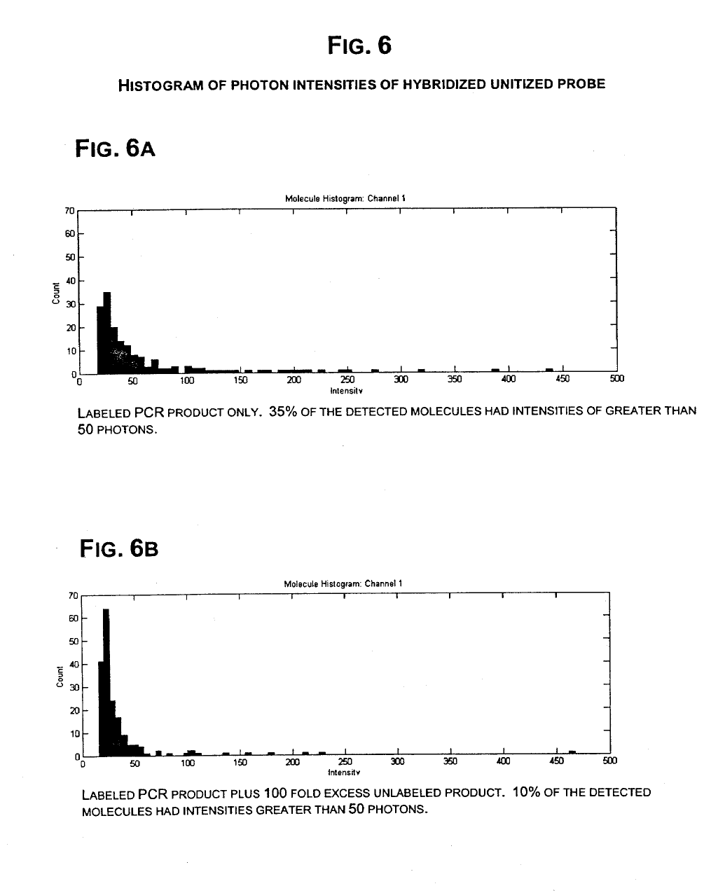

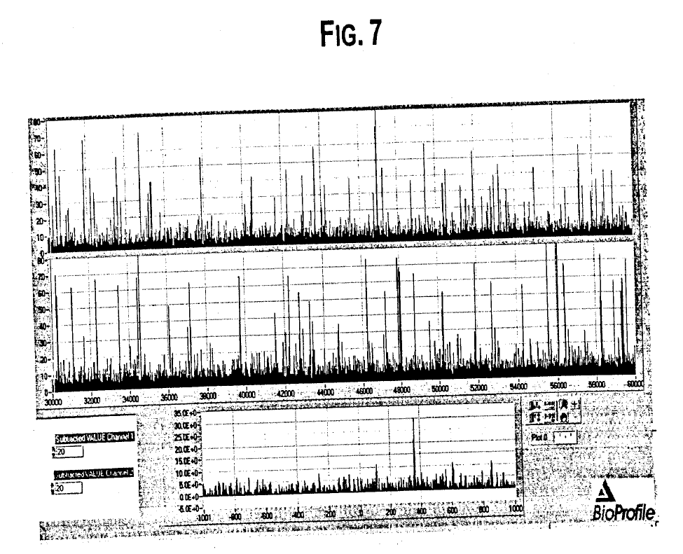

Molecules are pumped through the capillary, or an electric field is applied to the sample to move molecules electrophoretically. Under electrophoretic conditions, like molecules move through the tube in lockstep (plug flow). As molecules pass through each laser beam, excitation of each fluorescent molecule takes place via one-photon excitation. Within a fraction of a second, the excited molecule relaxes, emitting a detectable burst of light. The excitation-emission cycle is repeated many times by each molecule in the length of time it takes for it to pass through the laser beam allowing the instrument to be able to detect hundreds of molecules per second. Photons emitted by fluorescent molecules are registered in both detectors with a time delay indicative of the time for the molecule (or molecular hybridization complex) to pass from the interrogation volume of one detector to the interrogation volume of the second detector. The photon intensity recorded by the detectors is then cross-correlated using a personal computer with a digital correlator card. The computer then produces a histogram of velocities that shows a peak for every fluorescent species present in the sample. When the sample is pumped through the capillary, all molecules move at the same velocity. When an electric field is applied to the sample, the transit time between the detectors for each molecule is dependent upon the molecule's characteristic charge, size and shape (Castro and Al. 1995; Castro and Shera 1995; Castro and Williams 1997). An example of the output of the existing laboratory system is shown in FIG. 3.

With the current instrument configuration (5 μm laser beam) approximately 0.25% of the fluorescent molecules in the solution pass through the laser beams and are detectable. This percentage can be increased by configuring each laser beam such that it forms a narrow band perpendicular to the length of the capillary (as shown in FIG. 2). Such an arrangement can raise the percentage of detectable molecules to approximately 5% of the molecules in the solution. Other configurations illuminating larger areas of the capillary have been calculated to enable detection of up to 50% of the fluorescent molecules present in a sample.

### III. Methods of Use

The present invention further encompasses methods of using probes, as well as compositions comprising a plurality of probes, and wherein each probe has a distinctive ratio of charge to translational frictional drag, to detect and characterize one or more selected nucleotide sequences within one or more target nucleic acids.

In one aspect, the present invention provides a method of detecting a plurality of sequences within one or more target nucleic acids, comprising contacting a plurality of probes, wherein each probe has a structure independently, with one or more target nucleic acids, generally under conditions that distinguish those that hybridize to the target nucleic acid, and detecting those which have hybridized to the target nucleic acid.

In one aspect of this method, the target nucleic acids are immobilized. In this aspect, the immobilized target nucleic acids are contacted with sequence-specific probes, which further comprise a detectable label, under conditions that distinguish those probes having sufficient homology to hybridize to the target nucleic acid. The non-hybridized probes are washed away and hybridized probes are recovered and detected after denaturation of the base-paired structure formed between the sequence-specific probe and the immobilized target nucleic acid.

In another aspect of this method, the target nucleic acid, which may be immobilized, is contacted with a plurality of sequence-specific probe probes whereby two probes hybridize to adjacent sequences of the target nucleic acid such that the 5′-end of one probe, which generally will carry a 5′-phosphate moiety, abuts the 3′-end of the second probe, so that the two probes can be covalently joined to one another, in certain embodiments, with a DNA chemical or enzymatic ligating activity, to form a ligated product. In this aspect of the method, the ligated product is formed by the joining of two probes, at least one of which comprises a detectable label and at least one of which is a sequence-specific probe, such that the ligated product has a distinctive ratio of charge to translational frictional drag. In a further aspect, three or more probes are hybridized to adjacent sequences of a target nucleic acid in such a manner that at least three probes can be covalently joined to form a ligated product, wherein at least one of the probes so joined comprises a detectable label, and at one of the probes so joined is a sequence-specific such that the ligated product has a distinctive ratio of charge to translational frictional drag. Generally, the ligated product, which is hybridized to the target nucleic acid, is released by denaturation, and the ligated product having a distinctive ratio of charge to translational frictional drag, is detected and analyzed, to provide information about the selected nucleotide sequence within the target nucleic acid.

This cycle of hybridization, joining, and denaturation, may be repeated in order to amplify the concentration of the ligated product formed. In this instance, the joining is optionally accomplished by means of a thermostable ligating enzyme. These reactions are conveniently carried out in thermal cycling machines with thermally stable ligases.

Furthermore, additional probes, which together are sufficiently complementary to the ligated product to hybridize thereto and be covalently joined to one another as above, are also included, thereby affording geometric amplification of the ligated product, i.e., a ligase chain reaction (Wu, D. Y. and Wallace B. (1989), The ligation amplification reaction (LAR)-Amplification of Specific DNA sequences using sequential Rounds of Template Dependent Ligation, Genomics 4:560-569; Barany, (1991), Proc. Natl. Acad. Sci. USA, 88:189; Barany, (1191), PCR Methods and Applic., 1:5). To suppress unwanted ligation of blunted ended hybrids formed between complementary pairs of the and second oligonucleotides and the second pair of oligonucleotides, conditions and agents inhibiting blunted ended ligation, for example 200 mM NaCl and phosphate, are included in the ligation reaction.

The product of such a ligase chain reaction therefore is a double stranded molecule consisting of two strands, each of which is the product of the joining of at least two sequence-specific probes. Accordingly, in yet another aspect of the present invention, at least one of the incorporated within the ligase chain reaction product comprises a detectable label, and at one of the is a sequence-specific probe such that the ligase chain reaction product has a distinctive ratio of charge to translational frictional drag.

In another aspect of the oligonucleotide ligase assays described above, mismatches, i.e. non-complementary nucleobases, existing between selected nucleotide sequences within the target nucleic acid and either or both of the sequence-specific probe and the second oligonucleotide interfere with the ligation of the two probes either by preventing hybrid formation or preventing proper joining of the adjacent terminal nucleotide residues. Thus, when the binding conditions are chosen to permit hybridization of both probes despite at least one mismatch, the formation of a ligated product reveals the sequence of the selected nucleotide sequence as it exists within the target nucleic acid, at least with respect to the terminal, adjacent residues of the two probes. Those skilled in the art are well versed in selecting appropriate binding conditions, such as cation concentration, temperature, pH, and oligonucleotide composition to selectively hybridize the probes to the selected nucleotide sequences within the target nucleic acid.

Since the base pairing of terminal adjacent residues affects ligation, in one embodiment the probe providing the 3′ terminal nucleobase involved in the joining reaction is designed to be perfectly complementary to the target sequence while the probe providing the 5′ terminal nucleobase residue involved in the joining reaction is designed to be perfectly complementary in all but the 5′ terminal nucleobase. In another embodiment, the probes are designed such that the probe providing the 3′ terminal nucleobase is perfectly complementary except for the 3′ terminal nucleobase residue while the oligonucleotide providing the 5′ terminal nucleobase is perfectly complementary (Wu, D. Y. and Wallace, B., (1989), Specificity of nick-closing activity of bacteriophage T4 DNA ligase, Gene 76: 245-254; Landegren, U. et al. (1988) A ligase mediated gene detection technique, Science 2241:1077-1080).

In a modification of the method set forth above, the sequence-specific probe comprises a nucleobase sequence that is complementary to the target sequence, but comprises a non-terminal mismatch with respect to non-target sequences. In this aspect of the invention, the composition of the sequence-specific probe and the nature of the experimental conditions are such that the probe will only hybridize to the target sequence. In this embodiment for example, a second probe that hybridizes to the target nucleobase, either upstream or downstream of the hybridized sequence-specific probe, may be ligated to that probe to form the ligated, product that is diagnostic of the presence of the target nucleotide sequence.

In a further modification of the embodiment set forth above, the sequence-specific probe is hybridized to a selected nucleotide sequence within a target nucleic acid that is immediately adjacent to the site of interest. A second sequence-specific probe is hybridized to the selected region within the target nucleic acid such that the hybridized oligonucleotides are separated by a gap of at least one nucleotide residue. In another embodiment, the length of the gap is a single nucleotide residue representing a single polynucleotide polymorphism in the target nucleic acid. Following hybridization, the complex, which consists of the two probes hybridized to the target nucleic acid, is treated with a nucleic acid polymerase in the presence of at least one deoxyribonucleoside triphosphate. If the deoxyribonucleoside triphosphate(s) provided are complementary to the target polynucleotide's nucleotide residues which define the gap, the polymerase fills the gap between the two hybridized probes. Subsequent treatment with ligase joins the two hybridized oligonucleotides to form a ligated, product, which can, in one embodiment, be separated from the template by thermal dissociation, thereby providing a diagnostic product having a distinctive ratio of charge to translational frictional drag. This diagnostic product will generally comprise a reporter molecule, which may be included within either of the ligated probes, be attached to the one or more nucleobases added by the polymerizing activity, or be added subsequent to the covalent joining of the probes. By treating with polymerase in the presence of fewer than four nucleoside triphosphates, the nucleotide residues comprising the gap may be determined. Further amplification of ligated product is achieved by repeated cycles of denaturation, annealing, nucleic acid polymerase gap filling, and ligation in the presence of at least one of the nucleoside triphosphates.

If the treatment with nucleic acid polymerase occurs in the presence of one labeled nucleoside triphosphate or a mixture containing one labeled and 3 unlabeled nucleoside triphosphates, ligated products comprising at least one incorporated, labeled nucleoside are readily detected upon electrophoretic separation of the labeled ligated products. Modification of the nucleotide mixture to one having one labeled nucleoside triphosphate and three chain terminating nucleoside triphosphates suppresses unwanted ligation of oligonucleotides with incorrectly incorporated nucleotide residues.

Probes of the present invention are also useful as primers for nucleic acid sequence analysis by the chain termination method, a method well known to those skilled in the art. In one embodiment, probes are hybridized to target nucleic acid and extended by a nucleic acid polymerase in the presence of a mixture of nucleoside triphosphates and a chain terminating nucleoside triphosphate. The polymerase reaction generates a plurality of chain terminated nucleic acids fragments, which are separated, for example by capillary electrophoresis. Chain termination by the incorporated chain terminating nucleoside triphosphate identifies the 3′ terminal residue of the terminated nucleic acid fragment.

For the purposes of detecting the chain terminated species, various substituents of the nucleic acid fragments are amenable to conjugation with detectable reporter molecules. These include the nucleoside triphosphate precursors, including the chain terminators, incorporated into the nucleic acid. Detectable reporter molecules may be radioactive, chemiluminescent, bioluminescent, fluorescent, or ligand molecules. In one embodiment, the detectable label is a fluorescent molecule, for example fluorescein isothiocyanate, Texas red, rhodamine, and cyanine dyes and derivatives thereof. In another embodiment, the fluorescent dyes are to reduce the variations in electrophoretic mobility of nucleic acids caused by the fluorescent label (see e.g. Ju, J. et al. (1995). Design and synthesis of fluorescence energy transfer dye-labeled primers and their application for DNA sequencing and analysis, Anal. Biochem. 231: 131-40; Metzker, et al. (1996) Electrophoretically uniform fluorescent dyes for automated DNA sequencing, Science 271: 1420-22; Hung, S. C. et al. (1997) Comparison of fluorescence energy transfer primers with different donor-acceptor dye combinations, Anal Biochem. 252: 77-88; Tu, O. et al. (1998) The influence of fluorescent dye structure on the electrophoretic mobility of end-labeled DNA, Nucleic Acids Res. 26: 2797-2802)

In one embodiment, the of the present invention are used within a format for sequencing selected regions within a target polynucleotide wherein one of four spectrally resolvable fluorescent molecules is used to label the nucleic acid fragments in reactions having one of four chain terminating nucleoside triphosphates. In another aspect of this embodiment, the probes of the present invention are used for sequencing selected regions within a target polynucleotide wherein one of four spectrally resolvable fluorescent molecules is used to label an oligonucleotide primer in a reaction containing one of four chain terminating nucleoside triphosphates. Thus, in both aspects of this embodiment, detecting the fluorescent color of the chain terminated nucleic acid fragment identifies the 3′ terminal nucleotide residue. Separation of the chain-terminated products by electrophoresis, typically in a single gel lane or capillary, along with simultaneous on-line detection of four spectrally resolvable fluorescent molecules allows rapid sequence determination from the colors of the separated nucleic acid fragments (Prober, J. M. et al. (1985), A System for Rapid DNA Sequencing with Fluorescent Chain Terminating Dideoxynucleotides, Science 238: 336-341, Karger, A. E. et al., (1991), Multiwavelength Fluorescence Detection for DNA Sequencing Using Capillary Electrophoresis, Nucleic Acids Res. 19 (18):4955-62).

When the nucleotide sequence of interest is a small region of the target nucleic acid, for example a site including single nucleotide polymorphism, modified sequencing formats, optionally, are used. In one such embodiment, a sequence-specific probe is hybridized in a sequence-specific manner such that the 3′-terminal nucleotide residue of the sequence-specific probe is immediately adjacent to the site of interest. The hybridized probe is extended by a nucleic acid polymerase in the presence of at least one chain terminating nucleoside triphosphate extends the oligonucleotide by one nucleotide if the chain terminating nucleotide is complementary to the target nucleic residue immediately downstream of the 3′-terminus of the hybridized sequence-specific probe. Separation and detection of the extended, sequence-specific sequence-specific probe provides the identity of the residue immediately adjacent to the hybridized primer. In this embodiment, the use of a plurality of different probe permits the simultaneous detection and analysis of a plurality of target sequences in a single separation.

Detecting the extended primer is accomplished by including a reporter molecule conjugated to the extended, sequence-specific probes are used as primers in the same manner as described above for standard sequencing reactions. Thus, in one embodiment, the chain terminating nucleoside triphosphate is labeled with one of four spectrally resolvable fluorescent molecules such that the fluorescent label uniquely identifies the chain terminating nucleotide. The composition of the residue immediately adjacent to the hybridized oligonucleotide primer is then readily ascertained from the colors of the extended oligonucleotide primer. As will be apparent to those skilled in the art, this modified sequencing format is adaptable to other mixtures of fluorescently labeled chain terminating nucleoside triphosphates. Thus the embodiments encompass nucleotide combinations having two or four chain terminating nucleoside triphosphates wherein only one chain terminator is labeled with one of four resolvable reporter labels. Mixing the products of the extension reactions, followed by separation and detection of the extended products in a single gel lane or capillary provides the ability to determine all possible sequence variations at the nucleotide residue adjacent to the hybridized primer. Further increase in sensitivity of the methods are possible by using substantially exonuclease-resistant chain terminators, such as those which form thio-ester internucleotide linkages, to reduce removal of incorporated chain terminators by polymerase associated exonuclease.

In another embodiment, are used in polymerase chain reactions (PCR) to detect and amplify selected nucleotides within one or more target nucleic acids (Mullis, K., U.S. Pat. No. 4,683,202; Saiki, R. K., et al., Enzymatic Amplification of .beta.-Globin Genomic Sequences and Restriction Site Analysis for Diagnosis of Sickle Cell Anemia, In PCR: A practical approach, M. J. McPherson, P. Quirke, and G. R. Taylor, Eds., Oxford University Press, 1991). In this aspect of the present invention, the detection method involves PCR amplification of nucleotide sequences within the target nucleic acid. In this aspect, a target nucleic acid, which may be immobilized, is contacted with a plurality of 7 two of which hybridize to complementary strands, and at opposite ends, of a nucleotide sequence within the target nucleic acid. Repeated cycles of extension of the hybridized sequence-specific oligonucleotides, optionally by a thermo-tolerant polymerase, thermal denaturation and dissociation of the extended product, and annealing, provide a geometric expansion of the region bracketed by the two probes. The product of such a polymerase chain reaction therefore is a double-stranded molecule consisting of two strands, each of which comprises a sequence-specific probe. In this aspect of the present invention, at least one of the sequence-specific oligonucleotides is a sequence-specific probe such that the double stranded polymerase chain reaction product has a distinctive ratio of charge to translational frictional drag. The polymerase chain reaction product formed in this aspect of the invention further comprises a label, which may be incorporated within either of the sequence-specific probes used as primers, or it may be incorporated within the substrate deoxyribonucleoside triphosphates used by the polymerizing enzyme. In yet another aspect, the polymerase chain reaction product formed is analyzed under denaturing conditions, providing separated single stranded products. In this aspect, at least one of the single stranded products comprises both a label and a sequence-specific primer such that the single-stranded product derived from double stranded polymerase chain reaction product has a distinctive ratio of charge to translational frictional drag. As is well known in the art, such a single-stranded product may also be generated by carrying out the PCR reaction with limiting amounts of one of the two sequence-specific probes used as a primer. By using distinctive sequence-specific nucleic acids or probes as primers, the PCR reaction can detect many selected regions within one or more target polynucleotides in a single assay by allowing separation of one PCR product from another. Moreover, those skilled in the art will recognize that using various combinations of primers provides additional ways to generate distinctive PCR products. For example, a combination of a probe and a second primer pair in the PCR reaction generates a PCR product with a single strand. On the other hand, a combination of a probe and a second probe, which is also mobility-modified, generates a PCR product having both strands that are mobility-modified, thus distinguishing itself from the PCR product with one strand. Thus, by varying the type of mobility-modifying group and the nucleic acid strands that are mobility-modified, the embodiments enlarge the capacity to detect multiple target segments.

Detection of the PCR products may be accomplished either during electrophoretic separation or after an electrophoretic separation. Intercalating dyes such as ethidium bromide, ethidium bromide dimers, SYBR®Green, or cyanine dye dimers such as TOTO, YOYO and BOBO are available for post separation detection (Haugland, R. P. Handbook of Fluorescent Probes and Research Chemicals, 6.sup.th ed, Molecular Probes, Inc., 1996). Alternatively, the PCR products further comprise reporter molecules, including but not limited to radioactive, chemiluminescent, bioluminescent, fluorescent, or ligand molecules that permit detection either during or subsequent to an electrophoretic separation. Methods for labeling the PCR products follow the general schemes presented for labeling in other methods described infra.

Detecting a selected nucleotide sequence within a target nucleic acid by PCR amplification also encompasses identifying sequence variations within segments of the target nucleic acid. These variations include, among others, single nucleotide polymorphisms and polymorphisms in variable nucleotide tandem repeats (VNTR) and short tandem repeats (STR), such as those defined by sequence tag sites (STS). Identifying polymorphic loci are of particular interest because they are often genetic markers for disease susceptibility (see e.g. Gastier, J. M., (1995), Hum Mol Genet, 4(10):1829-36; Kimpton, C. P., (1993), Automated DNA profiling employing multiplex amplification of short tandem repeat loci, PCR Methods Appl., 3(1): 13-22). If the polymorphisms relate to variations in VNTR or STR sequences, direct analysis of PCR products without further treatment suffices for detecting polymorphisms since the products differ in nucleotide length. The presence of PCR products, however, expands the capability of the PCR analysis to detect multiple polymorphic loci in a single reaction.

If the polymorphisms relate to single nucleotide differences, the variations are detectable by conducting PCR reactions using primers designed to have mismatches with the selected nucleic acid sequence within a target nucleic acid. The presence of intentional mismatches within the duplex formed by hybridization of the primer and the selected nucleic acid sequence within the target nucleic acid affects the thermal stability of those duplex molecules, which is reflected in the T.sub.m of those structures and thus, under selected conditions, results in preferential amplification of one target segment as compared to another. Such allele-specific polymerase chain reactions permit identification of mutations in single cells, or tissues containing a low copy number of one selected nucleotide sequences amongst a high background of other nucleotide sequences within one or more target nucleic acids (Cha, R. S., (1993), Mismatch amplification mutation assay (MAMA): application to the c-H-ras gene., PCR Methods Appl., 2(1) 14-20; Glaab., W. E. et al., (1999), A novel assay for allelic discrimination that combines fluorogenic 5′ nuclease polymerase chain (TaqMan®) and mismatch amplification mutation., Mutat. Res. 430:1-12).

In yet another aspect, single nucleotide differences are distinguished through analysis of higher order conformations of single stranded nucleic acids that form in a sequence dependent manner. In this embodiment, single stranded nucleic acids are generated by dissociating the PCR products into single strands, or by preferentially amplifying one strand by using limiting amounts of one primer in the PCR reaction (i.e. single-sided PCR). Under selected conditions, the single stranded nucleic acids are allowed to form higher order structures by intramolecular hydrogen bonding of the single stranded nucleic acid. Those skilled in the art are well versed in defining such permissive conditions (i.e. temperature, denaturant concentration, pH, cation concentration etc.) for forming the higher order structures. These conformations, which are sequence dependent and which therefore can be extremely sensitive to single nucleotide changes, affect the electrophoretic mobility of the nucleic acid, and thus reveal variation in a selected nucleotide sequence within a target nucleic acid by their unique electrophoretic mobility profiles. To enhance formation of higher order structures modifications are introduced into the primers used for the PCR reactions. For example, a primer is engineered with additional bases complementary to a part of the selected nucleotide sequence within a target nucleic acid containing the sequence variation, such that higher order conformations form when the additional bases on the primer “snapback” or re-anneal to the normal sequence but not to variant sequences (Wilton, S. D. (1998), Snapback SSCP analysis: engineered conformation changes for the rapid typing of known mutations, Hum. Mutat. 11(3): 252-8). Since the reliability of detecting single nucleotide variations is affected by size of the single stranded probe, conformation analysis using, with each having a distinctive ratio of charge to translational frictional drag, permits detection of a plurality of selected nucleotide sequences within a target nucleic acid while maintaining the optimal length needed for forming higher order structures (Sheffield, V. C, (1993), The sensitivity of single stranded conformation polymorphism analysis for the detection of single base substitutions, Genomics 16 (2): 325-32).

In yet another aspect of the invention, probes are cleaved to detect selected nucleotide sequences within one or more target nucleic acids. In one such embodiment, probes are hybridized to selected nucleotide sequences within one or more target nucleic acids. In another embodiment, PCR products comprising at least one sequence-specific probe serve as substrates for sequence-specific enzymes, such as restriction enzymes. Digestion of the substrates by the enzymes creates cleaved products having a distinctive ratio of charge to translational frictional drag, which provides information about sequence composition of the target polynucleotides. This form of restriction fragment length polymorphism (RFLP) analysis is well known to those skilled in the art (see e.g., Kidd, I. M., (1998), A multiplex PCR assay for the simultaneous detection of human herpesvirus 6 and human herpesvirus 7, with typing of HHV-6 by enzyme cleavage of PCR products, J. Virol. Methods 70 (1): 29-36; Gelernter, J., (1991), Sequence tagged sites (STS) Taq I RFLP at dopamine beta-hydroxylase, Nucleic Acids. Res. 19 (8): 1957).

In another aspect, probes hybridized to selected nucleotide sequences within a target nucleic acid, wherein there is at least one nucleobase not complementary to the corresponding nucleobase in the target nucleic acid, are treated with agents that specifically cleave the non-base-paired nucleotide residues. Generally, the unpaired residue occurs on the hybridized probe (Bhattacharya, et al., (1989), Nucleic Acids. Res. 17, 6821-6840). Although chromosomal DNA may serve as the target nucleic acids, target nucleic acids are cloned DNA fragments comprising selected nucleotide sequences of a target nucleic acid, or PCR amplification products comprising selected nucleotide sequences of a target nucleic acid.

Cleavage may be accomplished with either chemical or enzymatic reagents. In chemical cleavage reactions, the hybrids containing at least one non-complementary nucleobase, are treated with chemicals which specifically modify the unpaired residue, rendering the internucleotide linkage of the modified nucleoside susceptible to hydrolysis. Suitable chemical agents include but are not limited to carbodiimide, osmium tetraoxide, hydroxylamine or potassium permanganate/tetraethylammonium chloride (Ellis, T. P., et al., (1998), Chemical cleavage of mismatch: a new look at an established method, Hum Mutat. 11: 345-53; Roberts, E., (1997), Potassium permanganate and tetraethylammonium chloride are safe and effective substitute for osmium tetraoxide in solid phase fluorescent chemical cleavage mismatch, Nucleic Acids. Res. 25: 3377-78). The use of potassium permanganate/tetraethylammonium chloride rather than osmium tetraoxide enhances cleavage at T/G mismatched pairs.

Enzymatic cleaving reagents encompass a variety of nucleases which recognize unpaired regions. These include but are not limited to single stranded specific nucleases such as S1 nuclease from Aspergillus oryzue, P1 from Penicillum citrinum, and mung bean nuclease (Shenk, et al., (1975) Proc. Natl. Acad. Sci. USA 72 989-93). Although these nucleases are less reactive towards single nucleotide mismatches, they can digest unpaired residues created by longer insertions and deletions (Dodgson, J. B. et al., (1977), Action of single-stranded specific nucleases on model DNA heteroduplexes of defined size and sequence, Biochemistry, 16:2374-49). Cel 1 and SP endonucleases show activity toward unpaired nucleotide residues resulting from nucleotides sequence variations comprising deletions, insertions, and missense mutations, within selected nucleotide sequences of target nucleic acids. (Oleykowski, C. A., (1998) Mutation detection using a novel plant endonuclease, Nucleic Acids. Res. 26: 4597-602; Yeung, A. T., U.S. Pat. No. 5,869,245). Resolvases from various sources, such as bacteriophage and yeast, represent yet another class of cleaving enzymes useful in this embodiment of the invention. Representative examples of resolvases include but are not limited to phage encoded T4 endonuclease VII and T7 endonuclease 1, both of which cleave at mismatches (Cotton, R. G. H., U.S. Pat. No. 5,958,692; Solaro, et al, (1993), Endonuclease VII of Phage T4 Triggers Mismatch Correction in vitro. J. Mol. Biol. 230: 868-877; (Chang, D. Y. et al., (1991), Base mismatch specific endonuclease activity in extracts from Saccharomyces cerevisiae; Nucleic Acids Research 19 (17): 4761-66).

In another aspect, of the present invention encompasses methods that prevent cleavage at unpaired residues. Proteins, including but not limited to the MutS protein of E. coli, bind to sites of single nucleotide mismatches in duplex nucleic acid structures (Su, S. S. et al., (1986), Escherichia coli mutS encoded protein binds to mismatched DNA base pairs, Proc. Natl. Acad. Sci. USA 83: 5057-5061). The MutS protein is part of the methylation directed E coli. MutH/S/L mismatch repair system, homologs of which are present in other bacteria, yeast and mammals (Eisen, J. A., (1998), A phylogenetic study of the MutS family of proteins, Nucleic Acids. Res. 26:4291-300; Alani, E. (1996), The Saccharomyces cerevisiae Msh2 and Msh6 proteins form a complex that specifically binds to duplex oligonucleotides containing mismatched DNA base pairs, Mol. Cell. Biol. 16: 5604-15; Modrich, P. et al., (1996), Mismatch repair in replication fidelity, genetic recombination and cancer biology, Annu. Rev. Biochem. 65:101-33). Therefore in one embodiment of the invention, duplex structures comprising at least one non-base-paired nucleobase unit formed by hybridization of a sequence-specific probe with a selected nucleotide sequence within a target nucleic acid, are treated with mismatch binding proteins such as MutS and then exposed to one or more exonucleases which degrade the duplex strands in a unidirectional fashion. A bound mismatch binding protein inhibits further action of the exonuclease on the strand containing the mismatch, thereby providing nucleic acid products of defined length and which possess a distinctive ratio of charge to translational frictional drag (Ellis, L. A., (1994), MutS binding protects heteroduplex DNA from exonuclease digestion in vitro: a simple method for detecting mutations, Nucleic Acids Res. 22 (13):2710-1; Taylor, G. R., U.S. Pat. No. 5,919,623). Unidirectional exonucleases suitable for use in this assay include, but are not limited to exonuclease III, bacteriophage lambda. exonuclease, and the 3′ to 5′ exonucleases of T7 DNA polymerase, T4 DNA polymerase, and Vent (tm) DNA polymerase.

In yet another aspect, a sequence-specific probe is used in a cleavage based method of detecting selected nucleotide sequences within a target nucleic acid may be a DNA-RNA-DNA probe, where an internal RNA segment is flanked by DNA segments. This tripartite sequence-specific probe is hybridized to a selected nucleotide sequence within a target nucleic acid at a temperature below the Tm of the overall, i.e. tripartite probe. Digestion of this duplex structure with an appropriate RNase, hydrolyzes only the RNA portion of the DNA-RNA-DNA probe when hybridized to a DNA template. In one embodiment, the RNase is a thermo-stable RNase H (Bekkaoui, F., (1996), Cycling probe technology with RNase H attached to an oligonucleotide, Biotechniques, 20 (2): 240-8). If the temperature of the reaction maintained above the Tm of the flanking DNA segments remaining after digestion of the internal RNA segment, those DNA segments dissociate, thus allowing another DNA-RNA-DNA oligomer to associate with the target polynucleotide. Repeated hybridization, RNA cleavage, and dissociation of the flanking DNA segments amplifies the level of detectable dissociated DNA segments. The reaction temperature, in one embodiment, is held constant during the amplification process, thus obviating any need for thermal cycling (Duck, P. (1990), Probe amplifier system based on chimeric cycling oligonucleotides, Biotechniques 9:142-48; Modrusan, Z. (1998) Spermine-mediated improvement of cycling probe reaction, Mol. Cell. Probes 12:107-16). In this aspect of the present invention, the sequence-specific DNA-RNA-DNA probe used comprise at least one mobility-modifying polymer and at least one reporter molecule attached to either or both of the flanking DNA segments, thereby providing a labeled digestion product having a distinctive ratio of charge to translational frictional drag.

In another aspect, detection of selected nucleotide sequences within one or more target nucleic acids based on cleavage of a probe relies upon cleavage substrates formed by invasive hybridization, as described in Brow et al. U.S. Pat. No. 5,846,717. In this embodiment, the 5′-portion of a sequence-specific probe, which comprises a reporter molecule and which is hybridized to a target nucleic acid, is displaced by a second probe that hybridizes to the same region and thereby exposing that displaced sequence to cleavage with a cleaving reagent. In practicing the embodiment, the target nucleic acid is contacted with a sequence-specific probe and with a second probe. The sequence-specific probe has a 5′-segment complementary to a second region of the selected nucleotide sequence contained within a target nucleic acid and a 3′-portion complementary to a third region of the selected nucleotide sequence contained within a target nucleic acid, wherein the second region is downstream from the third region. The second probe has a 5′-segment complementary to a first region of the selected nucleotide sequence contained within a target nucleic acid and a 3′-segment complementary to the second region of the selected nucleotide sequence contained within a target nucleic acid, wherein the first region is downstream from the second region. Under selected conditions, hybrids form in which the sequenced specific probe and the second probe hybridize to the target polynucleotide such that the second probe displaces the 5′ portion of the hybridized sequence-specific probe, whereas the 3′ portion of the sequence-specific probe and the 5′ portion of the second probe remain annealed to the selected nucleotide sequence contained within a target nucleic acid. The displaced strand, which is a single stranded segment that is not base-paired corresponds to the 5′-end of the sequence-specific probe then serves as a substrate for cleavage nucleases, thus producing discrete digestion products having distinct ratios of charge to translational frictional drag that reflect presence of specific sequences on the target polynucleotide. Cleaving enzymes recognizing displaced strands are either naturally occurring nucleases or modified nucleases. Naturally occurring structure-specific nucleases include, but are not limited to Pyrococcus woesii FEN-1 endonuclease, thermostable Methoanococcus jannaschii FEN-1 endonucleases, yeast Rad2, and yeast Rad1/Rad10 complex (Kaiser et al., U.S. Pat. No. 6,090,606, Cleavage Reagents; Kaiser, et al. U.S. Pat. No. 5,843,669, Cleavage of nucleic acid using thermostable Methoanococcus jannaschii FEN-1 endonucleases). Other structure-specific enzymes suitable for the cleaving reaction are those derived from modifications of known nucleases and polymerases (Dahlberg et al., U.S. Pat. No. 5,795,763, Synthesis Deficient Thermostable DNA Polymerase; Dahlberg et al., U.S. Pat. No. 6,614,402, 5′ Nucleases Derived from Thermostable DNA Polymerase). Modified polymerase that lack polymerase activity but still retain 5′-nuclease activity, are also used as cleaving reagents. Another embodiment is directed toward the use of the mobility-modifying polymers of the present invention in “invader assays,” which are SNP-identifying procedures based upon flap endonuclease cleavage of structures formed by two overlapping probes that hybridize to a target nucleic acid (see e.g. Cooksey et al., 2000, Antimicrobial Agents and Chemotherapy 44: 1296-1301). Such cleavage reactions release products corresponding to the 5′-terminal nucleobase(s) of the “downstream” probe. Where those cleavage products are labeled and can be separated from the uncleaved probe, an invader assay can be used to discriminate single base differences in, for example, genomic sequences or PCR-amplified genomic sequences.

Attachment of the mobility-modifying polymers of the present invention to the labeled 5′-terminus of the downstream probe used in an invader assay provides detectably-labeled cleavage products with distinctive charge to translational frictional drag ratios. Accordingly, a plurality of SNP's are analyzed simultaneously using a plurality of sequence-specific downstream probes, wherein the sequence-specific downstream probes comprise a mobility-modifying polymer of the present invention attached to the labeled 5′-terminus, such that the labeled product generated by flap endonuclease cleavage at each SNP has a distinctive charge to translational frictional drag ratio.

In a further aspect of the invader assay, for example, the downstream probe, which carries a label and a first mobility-modifying polymer of the present invention attached to the 5′-terminus, further comprises a second mobility-modifying polymer attached to the 3′-terminus. The presence of the second mobility-modifying polymer increases the sensitivity of the invader assay by enhancing the difference between the electrophoretic mobility of the flap endonuclease generated product, comprising the 5′-terminus, label, and first mobility-modifying polymer, and the electrophoretic mobility of the uncleaved downstream probe. Accordingly, the second mobility-modifying polymer has a molecular weight of at least 2000. In other embodiments, the second mobility-modifying polymer has a molecular weight of at least 5,000, at least 10,000, at least 20,000, and at least 100,000. In one embodiment, the second mobility-modifying polymer is a mobility-modifying polymer of the present invention, while in other embodiments, the second mobility-modifying polymer is a mobility-modifying polymer of the art, which is, in one illustrative, non-limiting example, an uncharged mono methyl polyethyleneglycol polymer. Moreover, the second mobility-modifying polymer may comprise a mixture of species of different molecular weight, provided that those species do not interfere substantially with detection of the signal product, i.e., the flap endonuclease generated product, comprising the 5′-terminus, label, and first mobility-modifying polymer (see Example 5, below). More generally, in other embodiments of the present invention, invader assays are performed in which the downstream oligonucleobase polymer comprises a label and a mobility-modifying polymer of the present invention attached to a first region of the downstream oligonucleobase polymer, and a second, high-molecular weight mobility-modifying polymer attached to a second region of the downstream oligonucleobase polymer, wherein first and second regions are separated by the flap endonuclease cleavage site. One aspect of this embodiment is described above and in Example 5, wherein the label and mobility-modifying polymer of the present invention are attached to the 5′-end of the sequence-specific oligonucleobase polymer and a second, high molecular weight mobility-modifying polymer is attached to the 3′-end of the sequence-specific oligonucleobase polymer. In other embodiments, for example, a second, high molecular weight mobility-modifying polymer is attached, via a linker arm nucleotide residue, to the sequence-specific probe, rather than at the 5′-end or 3′-end of the sequence-specific probe. Accordingly, the second, high molecular weight mobility-modifying polymer, is attached at any nucleobase residue within the second region of the downstream probe, or to the 5′-end or 3′-end, whichever is included within the second region of the downstream oligonucleobase polymer. Similarly, in some embodiments, the label, which is a fluorescent dye in certain non-limiting examples, is also attached via a linker arm nucleotide residue at any nucleobase residue within the first region of the downstream probe. Synthesis of such linker arm nucleotides and the coupling of, inter alia, a fluorescent dye or an uncharged mono methyl polyethyleneglycol polymer to the linker, are within the scope of the art (see e.g., Section 4.5 above). Moreover, e.g., linker arm nucleoside phosphoramidite monomers, as well as linker arm nucleoside phosphoramidite monomers comprising fluorescent moieties, are commercially available (Glen Research, Inc., Sterling, Va.). In these embodiments, the mobility-modifying polymer of the present invention is attached to the first region of the downstream probe, where the point of attachment may be at the 5′-end or the 3′-end, whichever is encompassed within the first region of the downstream probe, or the mobility-modifying polymer of the present invention may be incorporated within the first region of the downstream probe, providing a molecule according to Structural formula (IV). Therefore, in each of these embodiments, the presence of the second high molecular weight mobility-modifying polymer attached to the second region of the downstream probe increases the sensitivity of the invader assay by enhancing the difference between the electrophoretic mobility of the flap endonuclease generated product comprising a label and a mobility-modifying polymer of the present invention, i.e., the first region of the downstream oligonucleobase polymer, and the electrophoretic mobility of the uncleaved downstream probe.

In a still further embodiment of an invader assay, the downstream probe carries a label and a first mobility-modifying polymer, which is in one non-limiting embodiment, a standard PEO mobility-modifying polymer of the art, that is attached to the first region of the downstream probe, and a second, high molecular weight mobility-modifying polymer attached to the second region of the downstream probe. As above, the presence of the second mobility-modifying polymer increases the sensitivity of the invader assay by enhancing the difference between the electrophoretic mobility of the flap endonuclease generated product, i.e., the first region of the downstream probe, which comprises a label and a first mobility-modifying polymer, and the electrophoretic mobility of the uncleaved downstream probe. Accordingly, the second mobility-modifying polymer has a molecular weight of at least 2000. In other embodiments, the second mobility-modifying polymer has a molecular weight of at least 5,000, at least 10,000, at least 20,000, and at least 100,000. In one embodiment, the second mobility-modifying polymer is a mobility-modifying polymer of the present invention, while in other embodiments, the second mobility-modifying polymer is a mobility-modifying polymer of the art, which is, in one illustrative, non-limiting example, an uncharged mono methyl polyethyleneglycol polymer.

In another aspect of the present invention the sequence-specific probe serves as a cleavage substrate in detection reactions involving multiple sequential cleavage reactions, as described in Hall, J. G. et al., U.S. Pat. No. 5,994,069. In this embodiment, a first cleavage structure is formed as set forth above, except that in the present embodiment, the first probe is optionally a sequence-specific probe. The reaction mixture further includes a second target nucleic acid and a third probe, which is a sequence-specific probe, and further comprises at least one attached reporter molecule. The second target polynucleotide has a first, a second and a third region, wherein the first region is downstream of the second region, and the second region is downstream of the third region. The third probe has a 5′ portion fully complementary to the second region of the second target polynucleotide and a 3′ portion fully complementary to the third region of the second target polynucleotide. Treatment of the first cleavage structure results in release of a fourth probe, which has a 5′ portion complementary to the first region of the second target polynucleotide and a 3′ portion fully complementary to the second region of the second target polynucleotide. This released fourth probe forms a cleavage structure with the second target polynucleotide and the third probe under conditions where the 3′ portion of the third probe and the 5′ portion of the fourth probe remains annealed to the second target polynucleotide. Cleavage of the third probe with a cleavage reagent generates a fifth and sixth probe, either or both of which comprise a reporter molecule and a mobility-modifying polymer, thereby providing a digestion product having a distinctive ratio of charge to translational frictional drag. The fifth probe is released upon cleavage, while the sixth probe remains hybridized to the second target polynucleotide until dissociated by denaturation. Subsequent separation and detection of the fifth or sixth probe provides information about the presence of the first and second selected nucleotide sequence within the target nucleic acid.

In a further aspect of the present invention relating to a nucleotide sequence detection method involving multiple sequential cleavage reactions, a first cleavage structure is formed by first and second probe and a selected nucleotide sequence within a target nucleic acid, as set forth above. This aspect of the method further comprises a sequence-specific second target probe, which has a first, a second, and a third region, wherein the first region is downstream of the second region, and wherein the third region upstream of the second region, is fully self complementary and also complementary to the second region, such that it forms a hairpin structure under selected conditions. Cleavage of the first cleavage structure with a cleaving reagent generates a fourth probe, which has a 5′-portion complementary to the first region and a 3′-portion fully complementary to the second region of the probe. Hybridization of the released fourth probe to the first and second regions of the sequence-specific probe forms a second cleavage structure with a displaced third region that is complementary to the second region. Cleavage of this second cleavage structure generates a fifth and sixth probes, either of which comprises a mobility-modifying polymer and a label, thereby providing s digestion product having a distinctive ratio of charge to translational frictional drag, and whose separation and detection provides information about the presence of the first target nucleic acid and the second probe.

Methods for labeling and detecting the cleaved probes, as set forth infra., are equally applicable to the labeling and detection of products of the cleavage reactions. Moreover, labeling of released cleavage products is also accomplished by extension of the product by template independent polymerases, including but not limited to terminal transferase and polyA polymerase as described in U.S. Pat. No. 6,090,606, which is hereby specifically incorporated by reference.

In yet another aspect, the probes of the present invention are employed within a general method to effect the electrophoretic analysis and/or separation of target nucleic acids of identical or different sizes in non-sieving media. Normally, nucleic acids of different length (i.e. consisting of different numbers of nucleobase residues) display an essentially invariant ratio of charge to translational frictional drag. Accordingly, such molecules cannot be separated electrophoretically in non-sieving media. However, attachment of a sequence-specific probe of the present invention to target nucleic acids of identical or different length alters their ratio of charge to translational frictional drag of the target nucleic acids in a manner and to a degree sufficient to effect their electrophoretic mobility and separation in non-sieving media. Furthermore, and in contrast to electrophoretic separations in sieving media, longer nucleic acids to which a sequence-specific probe of the present invention has been attached will migrate more rapidly than a shorter nucleic acid to which the same sequence-specific probe has been attached. Applicants believe, although without wishing to be held to that belief, that such separations are based upon the proportionately smaller effect of attachment of a mobility-modifying sequence-specific probe of defined mass and size to a longer chain nucleic acid molecule than to a shorter chain nucleic acid molecule. Consequently, the ratio of charge to frictional translational drag will be greater for the longer chain, providing the longer chain nucleic acid with a greater velocity in an electric field.

In yet a further aspect, to affect the electrophoretic analysis and/or separation of target nucleic acids sieving media can be employed. Attachment of a sequence-specific probe of the present invention to target nucleic acids of identical or different length alters their ratio of charge to translational frictional drag of the target nucleic acids in a manner and to a degree sufficient to effect their electrophoretic mobility and separation in sieving media providing additional emphasis on the size of the probe-target complex over any net charge affects of the association.

Attachment of a to a population of nucleic acids of different length can be accomplished using a variety of approaches, including but not limited to hybridization, crosslinking of hybridization complexes, enzymatic ligation or direct, synthetic incorporation of the mobility-modifying of the present invention into the population of nucleic acids of different lengths that are to be separated.

In one aspect of this method, a mobility-modifying sequence-specific probe is enzymatically ligated to a population of nucleic acids of different length but having a common nucleotide sequence at the 5′-end, as is seen within the products of a chain termination nucleic acid sequencing reaction or, effectively, in chemical cleavage sequencing reactions which are transparent to all sequences other than those comprising the labeled 5′-end of the nucleic acid substrate. In this embodiment a synthetic template oligonucleotide, having two distinct sequence regions would be used as a template to align the hybridized 3′-end of a mobility-modifying sequence-specific probe so that it would directly abut the hybridized 5′-end, which is generally phosphorylated, that is common to the population of nucleic acids to be separated, and permit the two molecules to be covalently joined. Therefore the 5′-region of the synthetic template oligonucleotide would consist of a nucleotide sequence complementary to the common 5′-end sequence of the molecules to be separated, while the 3′-region of the synthetic template would consist of sequences complementary to the 3′-end of the mobility-modifying sequence-specific probe to be joined. In another embodiment of this approach, the common 5′-end of the population of nucleic acids to be separated corresponds to that generated by a sequence-specific restriction endonuclease. Therefore the synthetic template nucleic acid consists of at least eight nucleobases, of which at least three would be complementary to a common 5′-sequence of the population of molecules to be separated. The design of such template nucleic acids, as well as the conditions under which the enzymatic joining of the hybridized target nucleic acid and the sequence-specific probe would be carried out, are well known to those of ordinary skill in the art. Accordingly, this embodiment of the invention is applicable to any population of molecules of different sizes, provided each has a common 5′-end sequence of at least three nucleotides, in certain embodiments, at least four nucleotides, and in further embodiments, at least eight nucleotides. Similar procedures, wherein the sequence common to a population of molecules of different sizes occurs at the 3′-end, and consequently, the mobility-modifying sequence-specific nucleic to be attached has a phosphorylated 5′-end with the mobility-modifying polymer attached to the 3′-end, are also included within the scope of the present invention.

In a further embodiment a mobility-modifying sequence-specific probe is synthesized or produced so as to be complementary to a nucleotide sequence within, for example, a sequencing vector, that is upstream of, i.e. toward the 5′-end of, the binding site of a sequencing primer used in Sanger, enzymatic chain termination sequencing reaction. In this embodiment, the sequence-specific probe is enzymatically ligated to the sequencing primer either before or after extension of the sequencing primer during a chain termination sequencing reaction. In this embodiment, the sequence-specific nucleic acid is synthesized so that, once hybridized to the template polynucleotide, its 3′-end would either directly abut the 5′-end of the hybridized sequencing primer, or that 3′-end would hybridize to sequences upstream of the 5′-end of the sequencing primer. In the latter instance, the resulting gap is filled with a nucleic acid polymerase and the extended molecule is then enzymatically ligated to the sequencing primer.

Another embodiment of the invention is related to the separation and/or analysis of probe and polynucleotides. Separation and/or analysis of oligonucleotides is effected by electrophoresis, chromatography, or mass spectroscopy. In methods employing electrophoresis, the format may be thin flat chambers. In another embodiment, the separation and/or analysis is carried out by electrophoresis in capillary tubes. In another embodiment, the separation and/or analysis is carried out by molecular electrophoresis. The advantages of capillary electrophoresis and/or molecular electrophoresis are efficient heat dissipation, which increases resolution and permits rapid separation under high electrical fields. Moreover, the small diameters and/or areas of the capillary tubes or other configurations used for molecular electrophoresis allow separation of numerous samples in arrays of capillaries.

Sieving or nonsieving media are applicable to separation of probes including but not limited to the reaction products generated in the detection methods disclosed herein. Sieving media include covalently crosslinked matrices, such as polyacrylamide crosslinked with bis-acrylamide (see e.g. Cohen, A. S. et al. (1988) Rapid separation and purification of oligonucleotides by high performance capillary gel electrophoresis, Proc. Natl. Acad. Sci. USA 85: 9660; Swerdlow, H. et al., (1990), Capillary gel electrophoresis for rapid, high resolution DNA sequencing, Nuc. Acids Res. 18 (6): 1415-1419) or linear polymers, for example hydroxypropylmethylcellulose, methyl cellulose, or hydroxylethylcellulose (Zhu et al. (1992), J. Chromatogr. 480: 311-319; Nathakamkitkool, S., et al. (1992), Electrophoresis 13:18-31).

In one embodiment, the electrophoretic medium is a non-sieving medium. Although polynucleotides are not readily separable in a non-sieving medium, probes and polynucleotides have distinctive ratios of charge to translational frictional drag that permit separation in a non-sieving media, even when the probe and polynucleotides are of the same length.

The method of claim 30 is shown in FIG. 1. Three probes are labeled with the same dye, but have different fluorescent intensities (in photons/msec) as listed in the figure. When the concentration of the probes is kept at a level such that only one probe species (molecule) is in the interrogation volume of the detector system, fluorescence does not exceed 70 photons/msec. Fluorescence above 70 photons/msec indicates that two probes are in the interrogation volume hybridized to the target molecule. As can be seen in the figure, the fluorescence intensity detected indicates the specific probes that are bound to the target.

### EXAMPLES

Without further elaboration, it is believed that one skilled in the art can, using the preceding description, utilize the present invention to its fullest extent. The following specific examples are offered by way of illustration and not by way of limiting the remaining disclosure.

Multi XI for detection of targets and quantitation of target sites: Detection of biotin sites on biotinylated nucleic acid molecules.

Electrophoresis of Monoclonal Mouse Anti-Biotin Ab/Zenon A647 Conjugates Bound to Multi-Biotinylated Nucleic Acid.

Methods: PhiX 174 viral DNA was used as a template to generate 2.6 kb PCR products in PBS buffer, pH 7.4 with and without incorporation of Biotin-16-dUTP during amplification. 1 ug of Monoclonal Mouse Anti-Biotin (IgG Fraction Monoclonal Mouse Anti-Biotin [IgG1K lsotype]1.3 mg/ml [Jackson ImmunoResearch, 200-002-096])

was mixed with Zenon Mouse IgG₁ Alexa 647 (Molecular Probes, Z025060) according to the procedures described in the Zenon product manual in the presence of PBS buffer, pH 7.4 and allowed to incubate at room temperature for 5 minutes. This reaction product was mixed with the biotinylated and non-biotinylated 2.6 kb PCR products in separate reactions in PBS buffer pH7.4. Reaction were run at Zenon-Ab: nucleic acid ratios of 1:10 and 30:1. Control incubations with Zenon alone and Ab alone also were done. All reaction mixtures were incubated for 1 hour at room temperature, then diluted prior to single molecule electrophoresis on the Singulex CoreMD instrument.

Results: Dye-labeled “Zenon” fragments (antibody fragments that bind specifically to the Fc regions of specific antibody subtypes) can be used as a means to label or detect target antibodies. In this embodiment of the invention, Zenon-labeled Anti-Biotin antibodies were used to detect the number of biotins on a multi-biotinylated nucleic acid. Each Zenon-labeled anti-biotin antibody generates about 5 photons in its transit through the interrogation volume of the CoreMD instrument (as seen in the control with labeled-antibody alone). The biotinylated DNA fragments mixed with Zenon-labeled anti-biotin antibody generate between 70-120 photons per molecule while in the instrument interrogation volume. The presence of the biotinylated DNA fragments was determined because multi-XI photon bursts were detected. (No increased photon burst size were detected with nonbiotinylated DNA target). By dividing the photon count detected for each biotinylated DNA molecule by the photon count/antibody it is possible to determine the number of antibodies bound per DNA molecule, which in turn indicates the number of biotins incorporated into each DNA molecule (assuming 100% binding and no steric hindrance). As many as 60 biotins were detected on a single DNA fragment.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A method for detecting a target nucleic acid molecule or target nucleic acid molecular complex comprising:
(a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and
(b) detecting interaction of the probes with the molecule or molecular complex, said interaction being detected by an increase in fluorescence intensity during a detection interval having a fluorescence intensity above the fluorescence intensity of any individual free probe, wherein molecule or molecular complex is analyzed such that only individual molecules or molecular complexes in contact with a probe are within an interrogation volume and within a detection time interval.

2. A method according to claim 1, wherein each probe in contact with the molecule or molecular complex possesses substantially equal fluorescence intensity.

3. A method according to claim 1, wherein each probe in contact with the molecule or molecular complex has a fluorescence intensity distinguishable from another probe in contact with the same molecule or molecular complex.

4. A method according to claim 1, wherein unbound probes are removed from contacting action (a) prior to detection.

5. A method according to claim 1, wherein during detection action (b) the concentration of unhybridized probes is maintained at a level such that the fluorescence intensity of unhybridized probes can be distinguished from the fluorescence intensity of the probes in contact with the molecule or molecular complex.

6. A method according to claim 1, wherein the probes are selected from the group consisting of DNA, RNA, PNA, LNA, XLNT, nucleic acid sequence-specific molecules, structure-specific molecules, and any combination thereof.

7. A method according to claim 1, wherein detection of probes in contact with the molecule or molecular complex is provided by a single fluorescence detector.

8. A method according to claim 1, wherein detection of probes in contact with the molecule or molecular complex is provided by two or more fluorescence detectors.

9. A method according to claim 1, wherein detection of probes in contact with the molecule or molecular complex is provided by a CCD.

10. A method for detecting a target nucleic acid molecule or target nucleic acid molecular complex comprising:
(a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and
(b) detecting interaction of the probes in contact with the molecule or molecular complex, said interaction being detected by an increase in fluorescence intensity during a detection interval having a fluorescence intensity above the fluorescence intensity of any individual free probe; and
(c) detecting the velocity of the probes in contact with molecule or molecular complex, in relation to an expected velocity for such a complex in a transport tube, wherein the velocity is imparted on the probes in contact with the molecule or molecular complex by pumping the probes in contact with the molecule or molecular complex through the tube or by applying an electric field to the probes in contact with the molecule or molecular complex.

11. A method according to claim 10, wherein the number of probes interacting with the molecule or molecular complex can be ascertained by measuring the fluorescence intensity during a detection interval together with the velocity of the probes in contact with the molecule or molecular complex in the transport tube.

12. A method according to claim 10, wherein the specific probes interacting with the molecule or molecular complex can be ascertained by measuring fluorescence intensity during a detection interval together with the velocity of the probes in contact with the molecule or molecular complex in a transport tube.

13. A method according to claim 10, wherein each probe in contact with the molecule or molecular complex possesses substantially equal fluorescence intensity.

14. A method according to claim 10, wherein each probe in contact with the molecule or molecular complex has a fluorescence intensity distinguishable from another probe in contact with the same molecule or molecular complex.

15. A method according to claim 10, wherein unbound probes are removed from contacting action (a) prior to detection.

16. A method according to claim 10, wherein during detection actions (b) and (c) the concentration of unhybridized probes is maintained at a level such that the fluorescence intensity of unhybridized probes can be distinguished from the fluorescence intensity of the probes in contact with the molecule or molecular complex.

17. A method according to claim 10, wherein the probes are selected from the group consisting of DNA, RNA, PNA, LNA, XLNT, nucleic acid sequence-specific molecules, structure-specific molecules, and any combination thereof.

18. A method according to claim 10, wherein detection of probes in contact with the molecule or molecular complex is provided by a single fluorescence detector.

19. A method according to claim 10, wherein detection of probes in contact with the molecule or molecular complex is provided by two or more fluorescence detectors.

20. A method according to claim 10, wherein detection of probes in contact with the molecule or molecular complex is provided by a CCD.

21. A method for detecting a target nucleic acid molecule or target nucleic acid molecular complex comprising:
(a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and
(b) detecting interaction of the probes with the molecule or molecular complex, said interaction being detected by a change during a detection interval in a fluorescence parameter selected from the group consisting of fluorescence lifetime, fluorescence polarization or FRET, wherein molecule or molecular complex is analyzed such that only individual molecules or molecular complexes in contact with a probe are within an interrogation volume and within a detection time interval.

22. A method according to claim 21, wherein the specific probes interacting with the target can be ascertained by the change in a fluorescence parameter during a detection interval.

23. A method according to claim 21, wherein the specific probes interacting with the target can be ascertained by the change in a fluorescence parameter during a detection interval together with the velocity of the target-probes complex in a transport tube, such molecular velocity imparted by pumping of sample through the tube or by application of an electric field to the sample.

24. A method according to claim 21, wherein each probe in contact with the molecule or molecular complex possesses substantially equal fluorescence parameter values.

25. A method according to claim 21, wherein each probe in contact with the molecule or molecular complex possesses a fluorescence parameter value different from another probe in contact with the same molecule or molecular complex.

26. A method according to claim 21, wherein unbound probes are removed from contacting action (a) prior to detection.

27. A method according to claim 21, wherein during detection action (b) the concentration of unhybridized probes is maintained at a level such that the fluorescence intensity of unhybridized probes can be distinguished from the fluorescence intensity of the probes in contact with the molecule or molecular complex.

28. A method according to claim 21, wherein the probes are selected from the group consisting of DNA, RNA, PNA, LNA, XLNT, nucleic acid sequence-specific molecules, structure-specific molecules, and any combination thereof.

29. A method according to claim 21, wherein detection of probes in contact with the molecule or molecular complex is provided by a single fluorescence detector.

30. A method according to claim 21, wherein detection of probes in contact with the molecule or molecular complex is provided by two or more fluorescence detectors.

31. A method according to claim 21, wherein detection of probes in contact with the molecule or molecular complex is provided by a CCD.

32. A method according to claim 1, wherein the number of probes interacting with the molecule or molecular complex can be ascertained by measuring the fluorescence intensity during a detection interval.

33. A method according to claim 1, wherein the specific probes interacting with the molecule or molecular complex can be ascertained by measuring fluorescence intensity during a detection interval.

32. A method for detecting a target nucleic acid molecule or target nucleic acid molecular complex comprising:
(a) contacting two or more probes complementary to the molecule or molecular complex, said molecule or molecular complex being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex; and
(b) detecting interaction of the probes in contact with the molecule or molecular complex, said interaction being detected by a change during a detection interval in a fluorescence parameter selected from the group consisting of fluorescence lifetime, fluorescence polarization or FRET, during a detection interval having a fluorescence intensity above the fluorescence intensity of any individual free probe; and
(c) detecting the velocity of the probes in contact with molecule or molecular complex, in relation to an expected velocity for such a complex in a transport tube, wherein the velocity is imparted on the probes in contact with the molecule or molecular complex by pumping the probes in contact with the molecule or molecular complex through the tube or by applying an electric field to the probes in contact with the molecule or molecular complex.

33. A method according to claim 32, wherein the specific probes interacting with the target can be ascertained by the change in a fluorescence parameter during a detection interval.

34. A method according to claim 32, wherein the specific probes interacting with the target can be ascertained by the change in a fluorescence parameter during a detection interval together with the velocity of the target-probes complex in a transport tube, such molecular velocity imparted by pumping of sample through the tube or by application of an electric field to the sample.

35. A method according to claim 32, wherein each probe in contact with the molecule or molecular complex possesses substantially equal fluorescence parameter values.

36. A method according to claim 32, wherein each probe in contact with the molecule or molecular complex possesses a fluorescence parameter value different from another probe in contact with the same molecule or molecular complex.

37. A method according to claim 32, wherein unbound probes are removed from contacting action (a) prior to detection.

38. A method according to claim 32, wherein during detection actions (b) and (c) the concentration of unhybridized probes is maintained at a level such that the fluorescence intensity of unhybridized probes can be distinguished from the fluorescence intensity of the probes in contact with the molecule or molecular complex.

39. A method according to claim 32, wherein the probes are selected from the group consisting of DNA, RNA, PNA, LNA, XLNT, nucleic acid sequence-specific molecules, structure-specific molecules, and any combination thereof.

40. A method according to claim 32, wherein detection of probes in contact with the molecule or molecular complex is provided by a single fluorescence detector.

41. A method according to claim 32, wherein detection of probes in contact with the molecule or molecular complex is provided by two or more fluorescence detectors.

42. A method according to claim 32, wherein detection of probes in contact with the molecule or molecular complex is provided by a CCD.

43. A method for determining the number of probes interacting with a target comprising:
(a) contacting two or more probes that interact specifically with the target, said target being labeled with one or more fluorescent dye molecules of the same dye or labeled with two dyes that are indistinguishable by their emission characteristics in an assay instrument, wherein each probe interacts specifically with a different target nucleic acid sequence or a structure on the molecule or molecular complex, and wherein fluorescent intensity of each probe is equal to that of other probes within detection capabilities of an instrument used for the detection; and
(b) detecting interaction of the probes in contact with the target, said interaction being during a detection interval that is above the fluorescence intensity for any individual free probe molecule;
(c) detecting the velocity of the probes in contact with the target, in relation to an expected velocity for such a complex in a transport tube, wherein the velocity of the molecular probes-target hybrids matches the expected velocity for such a complex in a transport tube, and wherein the velocity is imparted on the probes in contact with the molecule or molecular complex by pumping the probes in contact with the molecule or molecular complex through the tube or by applying an electric field to the probes in contact with the molecule or molecular complex; and
(d) determining the number of probes interacting with each target molecule by dividing the fluorescent intensity detected per detection time interval by the unit intensity per detection time interval.

44. A method according to claim 43, wherein the number of free probe molecules (n) in the interrogation volume is greater than one, and the number of probes (greater than n) interacting with each target is determined by measuring the fluorescence intensity per detection time interval (I) and calculating the quantity (I/U)−n.

45. A method according to claim 43, wherein when the probes are labeled with luminescent dye(s) and the number of probes interacting with the target is be ascertained by the change in a luminescent parameter during a detection interval.

46. A method according to claim 45, wherein the luminescent parameter is selected from the group consisting of luminescence intensity, luminescence spectral distribution, burst size, burst duration, fluorescence lifetime, fluorescence polarization, FRET, and any combination thereof.

47. A method according to claim 45, wherein when the probes are labeled with luminescent dye(s) and the number of probes interacting with the target is ascertained by the change in a luminescent parameter during a detection interval together with the velocity of the target-probes complex in a transport tube, such molecular velocity imparted by pumping of sample through the tube or by application of an electric field to the sample.

48. A method according to claim 47, wherein the luminescent parameter is selected from the group consisting of luminescence intensity, fluorescence lifetime, fluorescence polarization, FRET, and any combination thereof.

49. A method according to claim 43, wherein the target is a nucleic acid and the probes are selected from the group consisting of nucleic acids, PNAs, LNAs, XLNT probes, peptides, proteins, small molecules, and any combination thereof.

50. A method for detecting a target nucleic acid molecule or target nucleic acid molecular complex comprising:
(a) contacting two or more probes that interact specifically with the target, said probe species being labeled with one or more fluorescent dye molecules of the same dye or labeled with one or more dye molecules of at least one other dye that is indistinguishable from the first dye by their emission characteristics in the assay instrument, wherein probe labeling takes place to the extent that luminescent or fluorescent intensity per detection time unit of each probe species is unique, and wherein each probe species is differentiable from the other probe species in the assay based on luminescent or fluorescent intensity per detection time unit; and
(b) detecting interaction of the probes in contact with the target, such that only individual target or probe molecules are within an interrogation volume and within a detection time interval and no more than one probe molecule is in the interrogation volume within a detection time interval, wherein specific probe species with target is detected by an increase in fluorescence intensity during a detection interval that is equal to the additive fluorescence intensities of more than one probe species; or
(c) detecting interaction of the probes in contact with the target, such that only individual target or probe molecules are within an interrogation volume and within a detection time interval and no more than one probe molecule is in the interrogation volume within a detection time interval, wherein specific multiple probe species with target is detected by an increase in fluorescence intensity during a detection interval that is equal to the additive fluorescence intensities of more than one probe species and the velocity of the molecular probes-target hybrids matches the expected velocity for such a complex in a transport tube, such molecular velocity imparted by pumping of sample through the tube or by application of an electric field to the sample.

51. A method according to claim 50, wherein the probes are labeled with luminescent dye(s) and the number of probes interacting with the target is ascertained by the change in a luminescent parameter during a detection interval.

52. A method according to claim 51, wherein the luminescent parameter is selected from the group consisting of luminescence intensity, luminescence spectral distribution, burst size, burst duration, fluorescence lifetime, fluorescence polarization, FRET, and any combination thereof.

53. A method according to claim 50, wherein the probes are labeled with luminescent dye(s) and the number of probes interacting with the target is ascertained by the change in a luminescent parameter during a detection interval together with the velocity of the target-probes complex in a transport tube, such molecular velocity imparted by pumping of sample through the tube or by application of an electric field to the sample.

54. A method according to claim 53, wherein the luminescent parameter is selected from the group consisting of luminescence intensity, luminescence spectral distribution, burst size, burst duration, fluorescence lifetime, fluorescence polarization, FRET and any combination thereof.

55. A method according to claim 50, wherein the target is a nucleic acid and probes are selected from the group consisting of nucleic acids, PNAs, LNAs, XLNT probes, peptides, proteins, small molecules, and any combination thereof.
